(19) United States
(12) Patent Application Publication
(10) Pub. No.: US 2024/0192218 A1
(43) Pub. Date: Jun. 13, 2024

(54) COMPOSITION AND KIT FOR DIAGNOSING CANCER AND METHOD FOR DIAGNOSING CANCER USING SAME

(71) Applicant: Attis Lab, Yuseong-gu Daejeon (KR)

(72) Inventor: Dong Ha BHANG, Dongjak-gu Seoul (KR)

(21) Appl. No.: 18/554,420

(22) PCT Filed: Apr. 8, 2022
(86) PCT No.: PCT/KR2022/005127

§ 371 Date: Oct. 6, 2023

(30) Foreign Application Priority Data
Apr. 9, 2021 (KR) 10-2021-0046621

(51) Int. Cl. G01N 33/574

(52) U.S. Cl. CPC G01N 33/57488

(57) ABSTRACT

Provided are a composition and a kit for diagnosing bladder cancer, breast cancer, cervical cancer, colorectal cancer, gastric cancer, liver cancer, lung cancer, ovarian cancer, pancreatic cancer, prostate cancer, and renal cancer, and a method for diagnosing cancer or a method for detecting a biomarker for diagnosing cancer, using the same. The present invention may be used as a composition or a kit for point-of-care testing-type diagnosis, and may diagnose bladder cancer, breast cancer, cervical cancer, colorectal cancer, gastric cancer, liver cancer, lung cancer, ovarian cancer, pancreatic cancer, prostate cancer, and renal cancer with high accuracy, sensitivity, and specificity.

### TECHNICAL FIELD

The present invention relates to a composition and a kit for diagnosing cancer, and a method for diagnosing cancer or a method for detecting a biomarker for diagnosing cancer, using the same.

### BACKGROUND ART

The World Health Organization (WHO) said that one-third of cancers can be cured through early screening and early treatment. The causes of cancer are diverse, but the number of causes due not only to genetic causes but also to the external environment is increasing. The causes of diseases caused by the external environment are so diverse that it is difficult to explain them with a single cause. Diseases caused by external environmental causes cannot be predicted and prevented in advance, and treatment is carried out only after the disease is diagnosed, so there are problems with cost, period, and considerable time required for recovery. Therefore, in order to diagnose cancer at an early stage, there is a need to develop a biomarker or a combination of biomarkers that can enhance the accuracy, sensitivity, and specificity for cancer diagnosis.

In addition, as the market for companion animals grows, there is a need to develop diagnosis technology for tumors, which are the number one cause of death in companion animals. Due to the high cost barrier of conventional diagnosis of malignant tumor and the lack of prompt diagnosis, the golden time for treatment can be missed. As a technology for point-of-care testing-type diagnosis in a companion dog disease, VDI's VDI-TKCANINE+, Sentinel Biomedical's CADET, VBTA (Veterinary Bladder Tumor Antigen) test method, Alertix Veterinary Diagnostics AB's Alertix Canine, and the like are commercially available. In Korea, a method for detecting cancer in dog has been developed in Korean Patent Application No. 10-2019-7001786 (Oct. 10, 2017).

Therefore, there is a need to develop a simple, rapid and accurate cancer diagnosis technology by developing a cancer biomarker or multiple biomarkers.

### DETAILED DESCRIPTION OF INVENTION

### Technical Problem

There is provided a composition for diagnosing bladder cancer, breast cancer, cervical cancer, colorectal cancer, gastric cancer, liver cancer, lung cancer, ovarian cancer, pancreatic cancer, prostate cancer, or renal cancer.

There is provided a kit for diagnosing bladder cancer, breast cancer, cervical cancer, colorectal cancer, gastric cancer, liver cancer, lung cancer, ovarian cancer, pancreatic cancer, prostate cancer, or renal cancer.

There is provided a method for diagnosing bladder cancer, breast cancer, cervical cancer, colorectal cancer, gastric cancer, liver cancer, lung cancer, ovarian cancer, pancreatic cancer, prostate cancer, or renal cancer, or a method for detecting a biomarker for diagnosis.

### Solution to Problem

In one aspect, there is provided a composition for diagnosing bladder cancer, comprising:

an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of canine TK1 (cTK1), CEA, AFP, and CRP.

The biomarker combination may further comprise one or more biomarkers selected from the group consisting of anti-human P53 (hP53) antibody, anti-c-myc antibody, human PKA (hPKA), PSA, B2M, and NGAL.

The biomarker combination may comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL;
(ii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M; and
(iii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M;
(ii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL;
(iii) anti-ECPKA antibody, anti-TK1 antibody, anti-granulin antibody, anti-Her-2 antibody, anti-hP53 antibody, anti-c-myc antibody, cTK1, AFP, and CRP;
(iv) anti-ECPKA antibody, anti-TK1 antibody, anti-granulin antibody, anti-Her-2 antibody, anti-hP53 antibody, anti-c-myc antibody, cTK1, AFP, and CEA;
(v) anti-ECPKA antibody, anti-TK1 antibody, anti-granulin antibody, anti-Her-2 antibody, anti-hP53 antibody, anti-c-myc antibody, cTK1, AFP, CEA, and PSA; and
(vi) anti-ECPKA antibody, anti-TK1 antibody, anti-granulin antibody, anti-Her-2 antibody, anti-hP53 antibody, anti-c-myc antibody, cTK1, AFP, CEA, PSA, and CRP.

The biomarker combination may further comprise a biomarker combination selected from the group consisting of:
(i) anti-ECPKA antibody, anti-TK1 antibody, anti-granulin antibody, and anti-Her-2 antibody;
(ii) anti-ECPKA antibody, anti-TK1 antibody, anti-granulin antibody, anti-Her-2 antibody, anti-hP53 antibody, and anti-c-myc antibody;
(iii) anti-ECPKA antibody, anti-TK1 antibody, anti-granulin antibody, anti-Her-2 antibody, anti-hP53 antibody, anti-c-myc antibody, and cP53;
(iv) anti-ECPKA antibody, anti-TK1 antibody, anti-granulin antibody, anti-Her-2 antibody, anti-hP53 antibody, anti-c-myc antibody, cP53, hPKA, and cTK1;
(v) anti-ECPKA antibody, anti-TK1 antibody, anti-granulin antibody, anti-Her-2 antibody, anti-hP53 antibody, anti-c-myc antibody, cP53, hPKA, cTK1, and AFP;
(vi) anti-ECPKA antibody, anti-TK1 antibody, anti-granulin antibody, anti-Her-2 antibody, anti-hP53 antibody, anti-c-myc antibody, cP53, hPKA, cTK1, AFP, and CEA;
(vii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, and PSA;
(viii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, and CRP;
(ix) anti-ECPKA antibody, anti-TK1 antibody, anti-granulin antibody, anti-Her-2 antibody, anti-hP53 antibody, and anti-c-myc antibody;
(x) anti-ECPKA antibody, anti-TK1 antibody, anti-granulin antibody, anti-Her-2 antibody, anti-hP53 antibody, anti-c-myc antibody, and cTK1; and
(xi) anti-ECPKA antibody, anti-TK1 antibody, anti-granulin antibody, anti-Her-2 antibody, anti-hP53 antibody, anti-c-myc antibody, cTK1, and AFP.

In another aspect, there is provided a composition for diagnosing breast cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of cTK1, AFP, B2M, and NGAL.

The biomarker combination may further comprise one or more biomarkers selected from the group consisting of anti-hP53 antibody, anti-c-myc antibody, cP53, hPKA, CEA, and CRP.

The biomarker combination may comprise the following biomarker combination:
anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL.

The biomarker combination may further comprise the following biomarker combination:
anti-ECPKA antibody, anti-TK1 antibody, anti-granulin antibody, anti-Her-2 antibody, anti-hP53 antibody, anti-c-myc antibody, cP53, hPKA, cTK1, AFP, CEA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL;
(ii) anti-granulin antibody, anti-Her-2 antibody, anti-hP53 antibody, anti-c-myc antibody, cP53, hPKA, cTK1, AFP, CEA, PSA, CRP, B2M, and NGAL;
(iii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL;
(iv) anti-ECPKA antibody, anti-TK1 antibody, anti-granulin antibody, anti-Her-2 antibody, TK1, AFP, B2M, and NGAL;
(v) anti-ECPKA antibody, anti-TK1 antibody, anti-granulin antibody, anti-Her-2 antibody, anti-hP53 antibody, anti-c-myc antibody, cP53, cTK1, AFP, CEA, CRP, B2M, and NGAL; and
(vi) anti-ECPKA antibody, anti-TK1 antibody, anti-granulin antibody, anti-Her-2 antibody, anti-hP53 antibody, anti-c-myc antibody, hPKA, cTK1, AFP, CEA, CRP, B2M, and NGAL.

In another aspect, there is provided a composition for diagnosing cervical cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of AFP and B2M.

The biomarker combination may further comprise one or more biomarkers selected from the group consisting of anti-hP53 antibody, anti-c-myc antibody, hPKA, cTK1, CEA, PSA, CRP, and NGAL.

The biomarker combination may comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M;
(i) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL;
(iii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M;
(iv) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL; and
(v) anti-ECPKA antibody, anti-TK1 antibody, anti-granulin antibody, anti-Her-2 antibody, AFP, and B2M.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL;
(ii) anti-granulin antibody, anti-Her-2 antibody, anti-hP53 antibody, anti-c-myc antibody, cP53, hPKA, cTK1, AFP, CEA, PSA, CRP, B2M, and NGAL;
(iii) anti-granulin antibody, anti-Her-2 antibody, anti-hP53 antibody, anti-c-myc antibody, cTK1, AFP, CEA, PSA, CRP, B2M, and NGAL; and
(iv) anti-ECPKA antibody, anti-TK1 antibody, AFP, and B2M.

The biomarker combination may further comprise the biomarker combination of anti-ECPKA antibody, anti-TK1 antibody, and B2M.

In another aspect, there is provided a composition for diagnosing colorectal cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of AFP, CEA, CRP, and NGAL.

The biomarker combination may further comprise one or more biomarkers selected from the group consisting of anti-hP53 antibody, anti-c-myc antibody, cTK1, PSA, and B2M.

The biomarker combination may comprise the biomarker combination of anti-ECPKA antibody, anti-TK1 antibody, anti-granulin antibody, anti-Her-2 antibody, AFP, CEA, CRP, and NGAL.

The biomarker combination may further comprise the biomarker combination of anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL;
(ii) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL; and
(iii) anti-ECPKA antibody, anti-TK1 antibody, anti-granulin antibody, anti-Her-2 antibody, AFP, and CEA.

In another aspect, there is provided a composition for diagnosing gastric cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and cTK1.

The biomarker combination may further comprise one or more biomarkers selected from the group consisting of cP53, hPKA, CEA, PSA, CRP, B2M, and NGAL.

The biomarker combination may comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M; and
(ii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, and hPKA; and
(ii) anti-ECPKA antibody, anti-TK1 antibody, anti-granulin antibody, anti-Her-2 antibody, anti-hP53 antibody, anti-c-myc antibody, cP53, hPKA, cTK1, and AFP.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, and cTK1;
(ii) anti-ECPKA antibody, anti-TK1 antibody, anti-granulin antibody, anti-Her-2 antibody, anti-hP53 antibody, anti-c-myc antibody, cP53, hPKA, cTK1, AFP, and CEA;
(iii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, and PSA;
(iv) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, and CRP;
(v) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL;
(vi) anti-ECPKA antibody, anti-TK1 antibody, anti-granulin antibody, anti-Her-2 antibody, anti-hP53 antibody, anti-c-myc antibody, cTK1, and AFP;
(vii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M;
(viii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL;
(ix) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL; and
(x) anti-ECPKA antibody, anti-TK1 antibody, anti-granulin antibody, anti-Her-2 antibody, anti-hP53 antibody, anti-c-myc antibody, AFP, and TK1.

In another aspect, there is provided a composition for diagnosing liver cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and cTK1.

The biomarker combination may further comprise one or more biomarkers selected from the group consisting of cP53, hPKA, CEA, PSA, and CRP.

The biomarker combination may comprise the biomarker combination of anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, and CRP.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, and AFP;
(ii) anti-ECPKA antibody, anti-TK1 antibody, anti-granulin antibody, anti-Her-2 antibody, anti-hP53 antibody, anti-c-myc antibody, cP53, hPKA, cTK1, AFP, and CEA;
(iii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, and PSA;
(iv) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL;
(v) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL;
(vi) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL; and
(vii) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M;
(ii) anti-ECPKA antibody, anti-TK1 antibody, anti-granulin antibody, anti-Her-2 antibody, anti-hP53 antibody, anti-c-myc antibody, cTK1, AFP, and CRP;
(iii) anti-granulin antibody, anti-Her-2 antibody, anti-hP53 antibody, anti-c-myc antibody, cP53, hPKA, cTK1, AFP, CEA, PSA, CRP, B2M, and NGAL;
(iv) anti-ECPKA antibody, anti-TK1 antibody, anti-granulin antibody, anti-Her-2 antibody, anti-hP53 antibody, anti-c-myc antibody, cTK1, AFP, CEA, PSA, and CRP;
(v) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M; and
(vi) anti-ECPKA antibody, anti-TK1 antibody, anti-granulin antibody, anti-Her-2 antibody, anti-hP53 antibody, anti-c-myc antibody, cTK1, AFP, and CRP.

In another aspect, there is provided a composition for diagnosing lung cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, and AFP.

The biomarker combination may further comprise one or more biomarkers selected from the group consisting of cP53 and cTK1.

The biomarker combination may comprise a biomarker combination selected from the group consisting of:
(i) anti-ECPKA antibody, anti-TK1 antibody, anti-granulin antibody, anti-Her-2 antibody, anti-hP53 antibody, and anti-c-myc antibody;
(ii) anti-ECPKA antibody, anti-TK1 antibody, anti-granulin antibody, anti-Her-2 antibody, anti-hP53 antibody, anti-c-myc antibody, and cP53;
(iii) anti-ECPKA antibody, anti-TK1 antibody, anti-granulin antibody, anti-Her-2 antibody, anti-hP53 antibody, anti-c-myc antibody, and cTK1; and
(iv) anti-ECPKA antibody, anti-TK1 antibody, anti-granulin antibody, anti-Her-2 antibody, anti-hP53 antibody, anti-c-myc antibody, and AFP.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M;
(ii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL;
(iii) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL;
(iv) anti-ECPKA antibody, anti-TK1 antibody, anti-granulin antibody, anti-Her-2 antibody, anti-hP53 antibody, anti-c-myc antibody, cTK1, AFP, CEA, PSA, and CRP;
(v) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M; and
(vi) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, and hPKA;
(ii) anti-ECPKA antibody, anti-TK1 antibody, anti-granulin antibody, anti-Her-2 antibody, anti-hP53 antibody, anti-c-myc antibody, cP53, hPKA, and cTK1;
(iii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, and CRP;
(iv) anti-ECPKA antibody, anti-TK1 antibody, anti-granulin antibody, anti-Her-2 antibody, anti-hP53 antibody, anti-c-myc antibody, cTK1, AFP, and CRP; and
(v) anti-ECPKA antibody, anti-TK1 antibody, anti-granulin antibody, anti-Her-2 antibody, anti-hP53 antibody, anti-c-myc antibody, AFP, CRP, and B2M.

In another aspect, there is provided a composition for diagnosing ovarian cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, CEA, CRP, and B2M.

The biomarker combination may further comprise one or more biomarkers selected from the group consisting of cP53, hPKA, cTK1, PSA, and NGAL.

The biomarker combination may comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M; and
(ii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M; and
(ii) anti-ECPKA antibody, anti-TK1 antibody, anti-granulin antibody, anti-Her-2 antibody, anti-hP53 antibody, anti-c-myc antibody, AFP, CEA, CRP, and B2M.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL;
(ii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL; and
(iii) anti-ECPKA antibody, anti-TK1 antibody, anti-granulin antibody, anti-Her-2 antibody, anti-hP53 antibody, anti-c-myc antibody, AFP, TK1, PSA, CEA, CRP, and B2M.

In another aspect, there is provided a composition for diagnosing pancreatic cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, cTK1, AFP, and CRP.

The biomarker combination may further comprise one or more biomarkers selected from the group consisting of CEA, PSA, B2M, and NGAL.

The biomarker combination may comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL; and
(ii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, and AFP;
(ii) anti-ECPKA antibody, anti-TK1 antibody, anti-granulin antibody, anti-Her-2 antibody, anti-hP53 antibody, anti-c-myc antibody, and cTK1; and
(iii) anti-ECPKA antibody, anti-TK1 antibody, anti-granulin antibody, anti-Her-2 antibody, anti-hP53 antibody, anti-c-myc antibody, cTK1, and AFP.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, and anti-c-myc antibody;
(ii) anti-ECPKA antibody, anti-TK1 antibody, anti-granulin antibody, anti-Her-2 antibody, anti-hP53 antibody, anti-c-myc antibody, cP53, hPKA, and cTK1;
(iii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M;
(iv) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL;
(v) anti-ECPKA antibody, anti-TK1 antibody, anti-granulin antibody, anti-Her-2 antibody, anti-hP53 antibody, anti-c-myc antibody, cTK1, AFP, and CRP;
(vi) anti-ECPKA antibody, anti-TK1 antibody, anti-granulin antibody, anti-Her-2 antibody, anti-hP53 antibody, and anti-c-myc antibody;
(vii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M; and
(viii) anti-ECPKA antibody, anti-TK1 antibody, anti-granulin antibody, anti-Her-2 antibody, TK1, AFP, and CRP.

In another aspect, there is provided a composition for diagnosing prostate cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and PSA.

The biomarker combination may further comprise one or more biomarkers selected from the group consisting of cTK1, CEA, CRP, B2M, and NGAL.

The biomarker combination may comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL; and
(ii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, and hPKA;
(ii) anti-ECPKA antibody, anti-TK1 antibody, anti-granulin antibody, anti-Her-2 antibody, anti-hP53 antibody, anti-c-myc antibody, cP53, hPKA, and cTK1;
(iii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M;
(iv) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL; and
(v) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, and cP53;
(ii) anti-ECPKA antibody, anti-TK1 antibody, anti-granulin antibody, anti-Her-2 antibody, anti-hP53 antibody, anti-c-myc antibody, cP53, hPKA, cTK1, and AFP;
(iii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, and PSA;
(iv) anti-ECPKA antibody, anti-TK1 antibody, anti-granulin antibody, anti-Her-2 antibody, anti-hP53 antibody, anti-c-myc antibody, cTK1, AFP, and CRP;
(v) anti-ECPKA antibody, anti-TK1 antibody, anti-granulin antibody, anti-Her-2 antibody, anti-hP53 antibody, anti-c-myc antibody, and cTK1;
(vi) anti-ECPKA antibody, anti-TK1 antibody, anti-granulin antibody, anti-Her-2 antibody, anti-hP53 antibody, anti-c-myc antibody, cTK1, and AFP;
(vii) anti-ECPKA antibody, anti-TK1 antibody, anti-granulin antibody, anti-Her-2 antibody, anti-hP53 antibody, anti-c-myc antibody, cTK1, AFP, and CEA;
(viii) anti-ECPKA antibody, anti-TK1 antibody, anti-granulin antibody, anti-Her-2 antibody, anti-hP53 antibody, anti-c-myc antibody, cTK1, AFP, CEA, and PSA;
(ix) anti-ECPKA antibody, anti-TK1 antibody, anti-granulin antibody, anti-Her-2 antibody, anti-hP53 antibody, anti-c-myc antibody, cTK1, AFP, and CRP;
(x) anti-granulin antibody, anti-Her-2 antibody, anti-hP53 antibody, anti-c-myc antibody, cTK1, AFP, CEA, PSA, CRP, B2M, and NGAL; and
(xi) anti-ECPKA antibody, anti-TK1 antibody, anti-granulin antibody, anti-Her-2 antibody, anti-hP53 antibody, anti-c-myc antibody, AFP, and PSA.

In another aspect, there is provided a composition for diagnosing renal cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, CEA, CRP, and B2M.

The biomarker combination may further comprise one or more biomarkers selected from the group consisting of cTK1, PSA, and NGAL.

The biomarker combination may comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL; and
(ii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M; and
(ii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M.

The biomarker combination may further comprise a biomarker combination selected from the group consisting of:
(i) anti-ECPKA antibody, anti-TK1 antibody, anti-granulin antibody, anti-Her-2 antibody, anti-hP53 antibody, anti-c-myc antibody, AFP, CEA, CRP, and B2M; and
(ii) anti-ECPKA antibody, anti-TK1 antibody, anti-granulin antibody, anti-Her-2 antibody, anti-hP53 antibody, anti-c-myc antibody, AFP, CEA, CRP, B2M, and NGAL.

In the anti-ECPKA antibody, the term “ECPKA” refers to extracellular protein kinase A. The protein kinase A (PKA) is also referred to as cAMP-dependent protein kinase. For humans, the ECPKA may comprise an amino acid sequence represented by Uniprot No. P17612. For dogs, the ECPKA may comprise an amino acid sequence represented by Uniprot No. Q8MJ44. The anti-ECPKA antibody may be an antibody specific for ECPKA or an antigen binding fragment thereof.

In the anti-TK1 antibody or TK1, the term “TK1” refers to thymidine kinase 1 (TK1). The TK1 may be cytosolic thymidine kinase 1 (cTK1). For humans, the TK1 may comprise an amino acid sequence represented by Uniprot No. P04183. For dogs, the TK1 may comprise an amino acid sequence represented by Uniprot No. E2QXP4. The anti-TK1 antibody may be an antibody specific for TK1 or an antigen binding fragment thereof.

In the anti-granulin antibody, the term “granulin” may also be referred to as GRN, CLN11, GEP, GP88, PCDGF, PEPI, PGranulin, or granulin precursor. The granulin may be in a cleaved form from its precursor, progranulin. The granulin may function in development, inflammation, cell proliferation, and protein homeostasis. For humans, the granulin may comprise an amino acid sequence represented by Uniprot No. P28799. For dogs, the granulin may comprise an amino acid sequence represented by Uniprot No. E2RHN1. The anti-granulin antibody may be an antibody specific for granulin or progranulin or an antigen binding fragment thereof.

In the anti-Her-2 antibody, the term “Her-2” refers to human epidermal growth factor receptor 2. The Her-2 may also be referred to as ERBB2, CD340, HER-2/neu, HER2, MLN 19, NEU, NGL, TKR1, or erb-b2 receptor tyrosine kinase 2. The Her-2 may be a member of the ErbB family, a growth factor receptor that promotes cell growth and division. The Her-2 may be overexpressed in breast cancer or gastric cancer. For humans, the Her-2 may comprise an amino acid sequence represented by Uniprot No. P04626. For dogs, the Her-2 may comprise an amino acid sequence represented by Uniprot No. Q18735. The anti-Her-2 antibody may be an antibody specific for Her-2 or an antigen binding fragment thereof.

In the anti-hP53 antibody, the term “hP53” refers to human P53. The P53 may also be referred to as TP53, BCC7, LFS1, P53, TRP53, tumor protein p53, or BMFS5. The P53 may be involved in the development of most cancers in the human body, including gastric cancer, colorectal cancer, prostate cancer, and lung cancer. The normal p53 gene has the function of inducing cell generation and apoptosis, but the p53 gene mutation may cause cancer by losing the ability to control cell growth and apoptosis. For humans, the P53 may comprise an amino acid sequence represented by Uniprot No. P04637. For dogs, the P53 may comprise an amino acid sequence represented by Uniprot No. Q29537 or E7FIY6. The anti-hP53 antibody may be an antibody specific for human P53 or an antigen binding fragment thereof.

In the anti-c-myc antibody, the term “c-myc” is also referred to as MYC, MRTL, MYCC, bHLHe39, c-Myc, v-myc avian myelocytomatosis viral oncogene homolog, MYC proto-oncogene, bHLH transcription factor, or myc. The c-myc may be a proto-oncogene encoding a transcription factor. The c-myc not only express factors that regulate cell cycle regulation and division, but may also play a role in function in cancer cell immune evasion mechanisms and cancer cell metabolic signals. For humans, the c-myc may comprise an amino acid sequence represented by Uniprot No. P01106. For dogs, the c-myc may comprise an amino acid sequence represented by Uniprot No. Q28350. The anti-c-myc antibody may be an antibody specific for c-myc or an antigen binding fragment thereof.

The AFP refers to alpha-fetoprotein. The AFP may also be referred to as AFPD, FETA, or HPalpha fetoprotein. The AFP may be a plasma protein found in a human fetus. The AFP is produced in the endoderm of the yolk egg in the early stages of development, but is produced in the liver in the later stage of development, and may decrease at birth, so that the concentration in the serum of adults is 10 ng/ml or less, and may increase again when a pathological condition occurs. For humans, the AFP may comprise an amino acid sequence represented by Uniprot No. P02771. For dogs, the AFP may comprise an amino acid sequence represented by Uniprot No. Q8MJU5.

The CEA refers to carcinoembryonic antigen. The CEA may be a glycoprotein involved in cell adhesion. The CEA has a low positive predictive value and is not often used as a screening test, but may be used to determine the stage, prognosis, and recurrence of colorectal cancer. For humans, the CEA may comprise an amino acid sequence represented by Uniprot No. P06731. For dogs, the CEA may comprise an amino acid sequence represented by Uniprot No. E2RPZ7.

The CRP refers to C-reactive protein. The CRP may be a ring-shaped pentameric protein present in plasma. The CRP may also be referred to as PTX1 (pentraxin-related). For humans, the CRP may comprise an amino acid sequence represented by Uniprot No. P02741. For dogs, the CRP may comprise an amino acid sequence represented by Uniprot No. E2R5J0.

The B2M refers to beta-2 microglobulin (β2 microglobulin). The B2M may be a component of MHC class I molecules. The B2M may also be referred to as IMD43. For humans, the B2M may comprise an amino acid sequence represented by Uniprot No. P61769. For dogs, the B2M may comprise an amino acid sequence represented by Uniprot No. A0A5F4C4Q1.

The NGAL refers to neutrophil gelatinase-associated lipocalin. The NGAL may be involved in innate immunity that sequesters iron and prevents its use by bacteria. The NGAL may also be referred to as LCN2, 24p3, MSFI, p25, or lipocalin 2. For humans, the NGAL may comprise an amino acid sequence represented by Uniprot No. P80188. For dogs, the NGAL may comprise an amino acid sequence represented by Uniprot No. E2RSM4.

The PSA refers to prostate-specific antigen. The PSA may be a protein present in male plasma. The PSA may also be referred to as gamma-seminoprotein, kallikrein-3, KLK3, APS, KLK2A1, PSA, hK3, or kallikrein-related peptidase 3. The PSA is secreted from the epithelial cells of the prostate, and may help sperm be ejaculated, and may serve to induce sperm into the uterus by dissolving vaginal mucus. The PSA is present in small amounts in healthy people, but its expression may increase in cases of prostate cancer or other prostate diseases. However, The PSA is not the only indicator of prostate cancer and may also diagnose prostatitis or benign prostatic hyperplasia. For humans, the PSA may comprise an amino acid sequence represented by Uniprot No. P07288. For dogs, the PSA may comprise an amino acid sequence represented by Uniprot No. P0728.

The cP53 refers to canine P53. The P53 is the same as described above.

The hPKA refers to human PKA. The PKA is the same as described above.

The fragment is a part of the protein and may be an immunogenic polypeptide.

The term “gene” is a unit of genetic information and refers to a nucleic acid sequence that encodes a substance having a function. The expression level of the gene may be the expression level of mRNA that encodes the protein. The expression level of mRNA may be a relative amount or an absolute amount of mRNA. Measuring the expression level of the gene may mean measuring the amount of mRNA.

The expression level of the protein or fragment thereof may be a relative amount or an absolute amount of the protein or fragment thereof. Measuring the expression level of the protein or fragment thereof may mean measuring the amount of the protein or fragment thereof.

The expression level may be increased compared to the expression level of a normal control group.

The agent may be an antibody or an antigen binding fragment thereof, or an aptamer that specifically binds to the protein or fragment thereof. The antibody may be a polyclonal antibody or a monoclonal antibody. The term “antibody” may be used interchangeably with the term “immunoglobulin.” The antibody may be a polyclonal antibody or a monoclonal antibody. The antibody may be a full length antibody. The antigen binding fragment refers to a polypeptide comprising an antigen binding site. The antigen binding fragment may be a single-domain antibody, Fab, Fab′, or scFv. The antibody or antigen binding fragment may be attached to a solid support. The solid support is, for example, the surface of a metal chip, plate, or well. The antibody or antigen binding fragment may be an antibody or an antigen binding fragment. The aptamer may be a single-stranded nucleic acid (DNA, RNA, or modified nucleic acid) or a peptide that specifically binds to the protein or fragment thereof.

The agent may be a nucleic acid comprising a polynucleotide identical to or complementary to a polynucleotide that encodes the protein or fragment thereof. The nucleic acid may be a primer, a probe, or an antisense oligonucleotide. The primer, probe, or antisense oligonucleotide may be labeled at its end or inside with a fluorescent substance, a chemiluminescent substance, or a radioactive isotope, etc.

The term “diagnosis” refers to determining the name of the disease, and may include the name of the cancer, the condition of the disease, the stage, the etiology, the presence or absence of complications, prognosis, and recurrence, etc.

In another aspect, there is provided a kit for diagnosing bladder cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of cTK1, CEA, AFP, and CRP.

In another aspect, there is provided a kit for diagnosing breast cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of cTK1, AFP, B2M, and NGAL.

In another aspect, there is provided a kit for diagnosing cervical cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of AFP and B2M.

In another aspect, there is provided a kit for diagnosing colorectal cancer: comprising
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of AFP, CEA, CRP, and NGAL.

In another aspect, there is provided a kit for diagnosing gastric cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and cTK1.

In another aspect, there is provided a kit for diagnosing liver cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and cTK1.

In another aspect, there is provided a kit for diagnosing lung cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, and AFP.

In another aspect, there is provided a kit for diagnosing ovarian cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, CEA, CRP, and B2M.

In another aspect, there is provided a kit for diagnosing pancreatic cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, cTK1, AFP, and CRP.

In another aspect, there is provided a kit for diagnosing prostate cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and PSA.

In another aspect, there is provided a kit for diagnosing renal cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, CEA, CRP, and B2M.

The kit may further comprise a sample necessary for cancer diagnosis. The kit may comprise a solid support, a substrate for immunological detection of an antibody or an antigen binding fragment, a suitable buffer solution, a chromogenic enzyme, a secondary antibody labeled with a fluorescent substance, or a chromogenic substrate. The kit may comprise polymerase, a buffering agent, nucleic acid, coenzyme, a fluorescent substance, or a combination thereof for detection of nucleic acid. The polymerase is, for example, Taq polymerase.

The kit may be a kit capable of digital quantification of signals.

In another aspect, there is provided a method for detecting a biomarker for diagnosing bladder cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of cTK1, CEA, AFP, and CRP in a biological sample isolated from a subject suspected of having bladder cancer; and
comparing the measured expression level with the expression level of a gene or the expression level of a protein or a fragment thereof in a normal control group.

In another aspect, there is provided a method for detecting a biomarker for diagnosing breast cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of cTK1, AFP, B2M, and NGAL in a biological sample isolated from a subject suspected of having breast cancer; and
comparing the measured expression level with the expression level of a gene or the expression level of a protein or a fragment thereof in a normal control group.

In another aspect, there is provided a method for detecting a biomarker for diagnosing cervical cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of AFP and B2M in a biological sample isolated from a subject suspected of having cervical cancer; and
measuring the expression level of a gene or the expression level of a protein or a fragment thereof selected from the group consisting of anti-ECPKA antibody, anti-TK1 antibody, anti-granulin antibody, anti-Her-2 antibody, AFP, and B2M; and
comparing the measured expression level with the expression level of a gene or the expression level of a protein or a fragment thereof in a normal control group.

In another aspect, there is provided a method for detecting a biomarker for diagnosing colorectal cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of AFP, CEA, CRP, and NGAL in a biological sample isolated from a subject suspected of having colorectal cancer; and
comparing the measured expression level with the expression level of a gene or the expression level of a protein or a fragment thereof in a normal control group.

In another aspect, there is provided a method for detecting a biomarker for diagnosing gastric cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and cTK1 in a biological sample isolated from a subject suspected of having gastric cancer; and
comparing the measured expression level with the expression level of a gene or the expression level of a protein or a fragment thereof in a normal control group.

In another aspect, there is provided a method for detecting a biomarker for diagnosing liver cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and cTK1 in a biological sample isolated from a subject suspected of having liver cancer; and
comparing the measured expression level with the expression level of a gene or the expression level of a protein or a fragment thereof in a normal control group.
   In another aspect, there is provided a method for detecting a biomarker for diagnosing lung cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, and AFP in a biological sample isolated from a subject suspected of having lung cancer; and
comparing the measured expression level with the expression level of a gene or the expression level of a protein or a fragment thereof in a normal control group.

In another aspect, there is provided a method for detecting a biomarker for diagnosing ovarian cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, CEA, CRP, and B2M in a biological sample isolated from a subject suspected of having ovarian cancer; and
comparing the measured expression level with the expression level of a gene or the expression level of a protein or a fragment thereof in a normal control group.

In another aspect, there is provided a method for detecting a biomarker for diagnosing pancreatic cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, cTK1, AFP, and CRP in a biological sample isolated from a subject suspected of having pancreatic cancer; and
comparing the measured expression level with the expression level of a gene or the expression level of a protein or a fragment thereof in a normal control group.

In another aspect, there is provided a method for detecting a biomarker for diagnosing prostate cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and PSA in a biological sample isolated from a subject suspected of having prostate cancer; and
comparing the measured expression level with the expression level of a gene or the expression level of a protein or a fragment thereof in a normal control group.

In another aspect, there is provided a method for detecting a biomarker for diagnosing renal cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, CEA, CRP, and B2M in a biological sample isolated from a subject suspected of having renal cancer; and
comparing the measured expression level with the expression level of a gene or the expression level of a protein or a fragment thereof in a normal control group.

The subject may be a mammal, such as a human, dog, cat, cow, horse, pig, sheep, goat, rabbit, mouse, hamster, hedgehog, ferret, or guinea pig. The subject may be a subject suffering from or suspected of having cancer, such as bladder cancer, breast cancer, cervical cancer, colorectal cancer, gastric cancer, liver cancer, lung cancer, ovarian cancer, pancreatic cancer, prostate cancer, and renal cancer.

The biological sample refers to a sample obtained from the subject. The biological sample may be blood, plasma, serum, bone marrow fluid, lymph fluid, saliva, tear fluid, mucosal fluid, amniotic fluid, or a combination thereof.

The measuring step may comprise the step of incubating the biological sample with an antibody or an antigen binding fragment thereof or an aptamer that specifically binds to the protein or fragment thereof, or incubating a polynucleotide identical to or complementary to a polynucleotide that encodes the gene.

The measuring step may be performed by electrophoresis, immunoblotting, enzyme-linked immunosorbent assay (ELISA), polymerase chain reaction, Northern blotting, polymerase amplification reaction (polymerase chain reaction: PCR), real-time polymerase chain reaction (real-time polymerase chain reaction or quantitative PCR: QPCR), reverse transcription polymerase chain reaction (rtPCR), protein chip, immunoprecipitation, microarray, electron microscopy, or a combination thereof. The electrophoresis may be SDS-PAGE, isoelectric electrophoresis, two-dimensional electrophoresis, or a combination thereof. The PCR may be real-time PCR or reverse transcription PCR.

The method comprises comparing the measured expression level with the expression level of a normal control group.

The expression level of the measured biomarker may be increased compared to the expression level of the biomarker derived from a sample of a normal control group. The normal control group refers to a negative control group that does not have cancer. In the method, the measured expression level of the biological sample may be increased compared to the expression level of a normal control group. For example, if the amount of the detected biomarker is greater than that of the normal control group, the subject may be diagnosed as having cancer or having a high probability of developing it. Anticancer therapy may be performed, such as administering an anticancer agent to a subject diagnosed with cancer.

### Effects of Invention

According to a composition and a kit for diagnosing cancer, and a method for diagnosing cancer or a method for detecting a biomarker for diagnosing cancer, using the same, according to one aspect, cancers such as bladder cancer, breast cancer, cervical cancer, colorectal cancer, gastric cancer, liver cancer, lung cancer, ovarian cancer, pancreatic cancer, prostate cancer, and renal cancer may be diagnosed at an early stage with high accuracy, sensitivity, and specificity.

### MODE FOR CARRYING OUT THE INVENTION

Hereinafter, the present invention will be described in more detail by way of examples. However, the following examples are only for illustrating the present invention, and the scope of the present invention is not limited to the examples.

### Example 1. Screening of Cancer Biomarkers

### 1. Screening Method for Cancer Biomarkers

About 3 mL to about 4 mL of the blood was obtained from patient dogs with cancer and normal dogs, and the serum was obtained therefrom (bladder cancer: 76 dogs, breast cancer: 50 dogs, cervical cancer: 10 dogs, colorectal cancer: 49 dogs, gastric cancer: 100 dogs, liver cancer: 50 dogs, lung cancer: 100 dogs, ovarian cancer: 22 dogs, pancreatic cancer: 59 dogs, prostate cancer: 119 dogs, renal cancer: 80 dogs). The obtained serum was separated and stored frozen or refrigerated until use.

In order to screen cancer biomarkers, kits that detect antigens in the blood and kits that detect antibodies were prepared (made in-house). The types of kits used and detection targets are summarized in Table 1 below.

TABLE 1

| Type of | | | | |
|---|---|---|---|---|
| target | | Location | | |
| protein | Kit name | in the kit | Target protein | Stationary phase |
| Antibody | cGHG | control | — | anti-canine IgG |
| | | group | | antibody |
| | | medium | anti-Her2 antibody | Her2 |
| | | bottom | anti-granulin antibody | granulin |
| | cGCP | control | — | anti-canine IgG |
| | | group | | antibody |
| | (P53/Cmyc) | medium | anti-c-myc antibody | canine c-myc antigen |
| | | bottom | anti-P53 antibody | canine P53 antigen |
| | cGET | control | — | anti-canine IgG |
| | | group | | antibody |
| | | medium | anti-cPKA antibody | cPKA antigen |
| | | bottom | anti-cTK1 antibody | cTK1 antigen |
| | cPKA | control | — | anti-canine IgG |
| | | group | | antibody |
| | | test group | anti-cPKA antibody | cPKA antigen |
| Antigen | cCRP | control | — | goat anti-mouse IgG |
| | | group | | |
| | | test group | cCRP | anti-cCRP antibody |
| | cTK1 | control | — | goat anti-mouse IgG |
| | | group | | |
| | | test group | Ctk1 | anti-Ctk1 antibody |
| | hAFP | control | — | goat anti-mouse IgG |
| | | group | | |
| | | test group | AFP | anti-AFP antibody |
| | cPKA | control | — | anti-chicken IgY |
| | | group | | antibody |
| | | test group | cPKA | anti-cPKA antibody |

In the kit name, the first letter “c” represents canine, and “h” represents human.

20 μL of separated serum that was frozen or refrigerated was left at room temperature for about 30 minutes. For each kit, a microtube (Axygen 1.5 mL Microtube) was inserted into the kit corresponding to the number of samples. As shown in Table 2 below, the buffer solution was added to the microtube according to the dilution factor and type of the buffer solution. The sample was diluted with the buffer solution and added to the microtube. BSB (borate saline buffered) or PB (phosphate buffer) was used as a buffer solution.

TABLE 2

| Kit name | cGET | cGHG | cGCP | cPKA | cCRP | cTK1 | hAFP | cPKA |
|---|---|---|---|---|---|---|---|---|
| Target | antibody | | | | antigen | | | |
| Dilution | 1:400 | 1:400 | 1:400 | 1:600 | 1:200 | 1:100 | 1:25 | 1:25 |
| factor | | | | | | | | |
| Serum | 1 μL | 1 μL | 1 μL | 1 μL | 1 μL | 2 μL | 5 μL | 5 μL |
| Buffer | 399 μL | 399 μL | 399 μL | 599 μL | 199 μL | 198 μL | 120 μL | 120 μL |
| Dilution | BSB | | | | | | | PB |
| buffer | | | | | | | | |
| Drop | 100 μL | 100 μL | 100 μL | 100 μL | 100 μL | 100 μL | 100 μL | 100 μL |
| amount of | | | | | | | | |
| solution | | | | | | | | |
| Travel | 12 min | | | | | | 10 min | 12 min |
| time | | | | | | | | |

The cTK1 antigen kit and the PKA antigen kit were instilled immediately after diluting the sample because the incubation time affects sensitivity.

Using MK4 equipment (made in-house, Bibox MK4), the absorbance (OD) of the spot detected in the kit was calculated.

### <MK4 Settings>

8 white chip LEDs
PWM (pulse width modulation) frequency: 100 Hz
PWM duty cycle: 50
Camera shutter exposure time: 0.4 seconds
Camera ISO: 100
Camera image color mode: YUV (obtained as Y value only)
Camera image resolution: 3264×2464

The cPKA kit and the cCRP kit used the V2 equipment (MPG consignment production, BT-AS2000). The V2 equipment changes color to purple when a reaction occurs in the kit, and measures the absorbance of the intensity of the purple part.

Additionally, kits for carcinoembryonic antigen (CEA), prostate-specific antigen (PSA), beta2-microglobulin (β2-microglobulin: B2M), and neutronphil gelatinase associated lipocalin (NGAL) were produced in-house and used.

By comparing the absorbance measured for each cancer type with the absorbance measured in the normal control group, a biomarker and a combination of biomarkers that were statistically significantly increased compared to the normal control group were selected. The accuracy, sensitivity, and specificity for diagnosis of bladder cancer, breast cancer, cervical cancer, colorectal cancer, gastric cancer, liver cancer, lung cancer, ovarian cancer, pancreatic cancer, prostate cancer, and renal cancer of the detected markers were calculated.

### 2. Bladder Cancer-Specific Biomarker

TABLE 3

| No. | Combination of Biomarkers | Accuracy | Sensitivity | Specificity |
|---|---|---|---|---|
| 1 | GET (anti-ECPKA antibody, anti-TK1 antibody) | 0.958 | 0.947 | 0.961 |
| 2 | GET (anti-ECPKA antibody, anti-TK1 antibody), GHG | 0.971 | 0.961 | 0.974 |
| | (anti-Gra antibody, anti-Her-2 antibody) | | | |
| 3 | GET (anti-ECPKA antibody, anti-TK1 antibody), GHG | 0.967 | 0.961 | 0.970 |
| | (anti-Gra antibody, anti-Her-2 antibody), GCP (anti-hP53 | | | |
| | antibody, anti-c-myc antibody) | | | |
| 4 | GET (anti-ECPKA antibody, anti-TK1 antibody), GHG | 0.971 | 0.961 | 0.974 |
| | (anti-Gra antibody, anti-Her-2 antibody), GCP (anti-hP53 | | | |
| | antibody, anti-c-myc antibody), cP53 | | | |
| 5 | GET (anti-ECPKA antibody, anti-TK1 antibody), GHG | 0.958 | 0.961 | 0.957 |
| | (anti-Gra antibody, anti-Her-2 antibody), GCP (anti-hP53 | | | |
| | antibody, anti-c-myc antibody), cP53, hPKA | | | |
| 6 | GET (anti-ECPKA antibody, anti-TK1 antibody), GHG | 0.964 | 0.947 | 0.970 |
| | (anti-Gra antibody, anti-Her-2 antibody), GCP (anti-hP53 | | | |
| | antibody, anti-c-myc antibody), cP53, hPKA, cTK1 | | | |
| 7 | GET (anti-ECPKA antibody, anti-TK1 antibody), GHG | 0.961 | 0.934 | 0.970 |
| | (anti-Gra antibody, anti-Her-2 antibody), GCP (anti-hP53 | | | |
| | antibody, anti-c-myc antibody), cP53, hPKA, cTK1, AFP | | | |
| 8 | GET (anti-ECPKA antibody, anti-TK1 antibody), GHG | 0.964 | 0.947 | 0.970 |
| | (anti-Gra antibody, anti-Her-2 antibody), GCP (anti-hP53 | | | |
| | antibody, anti-c-myc antibody), cP53, hPKA, cTK1, AFP, | | | |
| | CEA | | | |
| 9 | GET (anti-ECPKA antibody, anti-TK1 antibody), GHG | 0.961 | 0.934 | 0.970 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA | | | |
| 10 | GET (anti-ECPKA antibody, anti-TK1 antibody), GHG | 0.964 | 0.934 | 0.974 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP | | | |
| 11 | GET (anti-ECPKA antibody, anti-TK1 antibody), GHG | 0.977 | 0.961 | 0.983 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M | | | |
| 12 | GET (anti-ECPKA antibody, anti-TK1 antibody), GHG | 0.977 | 0.961 | 0.983 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M, NGAL | | | |
| 13 | GET (anti-ECPKA antibody, anti-TK1 antibody), | 0.987 | 0.974 | 0.991 |
| | GHG (anti-Gra antibody, anti-Her-2 antibody), GCP | | | |
| | (anti-hP53 antibody, anti-c-myc antibody), cTK1, | | | |
| | AFP, CEA, CRP, B2M, NGAL | | | |
| 14 | GET (anti-ECPKA antibody, anti-TK1 antibody), GHG | 0.980 | 0.961 | 0.987 |
| | (anti-Gra antibody, anti-Her-2 antibody), GCP (anti-hP53 | | | |
| | antibody, anti-c-myc antibody), cTK1, AFP, CRP | | | |
| 15 | GET (anti-ECPKA antibody, anti-TK1 antibody), AFP, | 0.961 | 0.961 | 0.961 |
| | CRP | | | |
| 16 | GET (anti-ECPKA antibody, anti-TK1 antibody), CRP | 0.958 | 0.947 | 0.961 |
| 17 | GHG (anti-Gra antibody, anti-Her-2 antibody), GCP | 0.961 | 0.974 | 0.957 |
| | (anti-hP53 antibody, anti-c-myc antibody), cP53, hPKA, | | | |
| | cTK1, AFP, CEA, PSA, CRP, B2M, NGAL | | | |
| 18 | GET (anti-ECPKA antibody, anti-TK1 antibody), GHG | 0.967 | 0.961 | 0.970 |
| | (anti-Gra antibody, anti-Her-2 antibody), GCP (anti-hP53 | | | |
| | antibody, anti-c-myc antibody) | | | |
| 19 | GET (anti-ECPKA antibody, anti-TK1 antibody), GHG | 0.974 | 0.961 | 0.978 |
| | (anti-Gra antibody, anti-Her-2 antibody), GCP (anti-hP53 | | | |
| | antibody, anti-c-myc antibody), cTK1 | | | |
| 20 | GET (anti-ECPKA antibody, anti-TK1 antibody), GHG | 0.967 | 0.934 | 0.978 |
| | (anti-Gra antibody, anti-Her-2 antibody), GCP (anti-hP53 | | | |
| | antibody, anti-c-myc antibody), cTK1, AFP | | | |
| 21 | GET (anti-ECPKA antibody, anti-TK1 antibody), GHG | 0.971 | 0.934 | 0.983 |
| | (anti-Gra antibody, anti-Her-2 antibody), GCP (anti-hP53 | | | |
| | antibody, anti-c-myc antibody), cTK1, AFP, CEA | | | |
| 22 | GET (anti-ECPKA antibody, anti-TK1 antibody), GHG | 0.977 | 0.947 | 0.987 |
| | (anti-Gra antibody, anti-Her-2 antibody), GCP (anti-hP53 | | | |
| | antibody, anti-c-myc antibody), cTK1, AFP, CEA, PSA | | | |
| 23 | GET (anti-ECPKA antibody, anti-TK1 antibody), GHG | 0.977 | 0.947 | 0.987 |
| | (anti-Gra antibody, anti-Her-2 antibody), GCP (anti-hP53 | | | |
| | antibody, anti-c-myc antibody), cTK1, AFP, CEA, PSA, | | | |
| | CRP | | | |
| 24 | GET (anti-ECPKA antibody, anti-TK1 antibody), | 0.987 | 0.974 | 0.991 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M | | | |
| 25 | GET (anti-ECPKA antibody, anti-TK1 antibody), | 0.987 | 0.974 | 0.991 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M, NGAL | | | |
| 26 | GHG (anti-Gra antibody, anti-Her-2 antibody), GCP | 0.958 | 0.987 | 0.948 |
| | (anti-hP53 antibody, anti-c-myc antibody), cTK1, AFP, | | | |
| | CEA, PSA, CRP, B2M, NGAL | | | |
| 27 | anti-ECPKA antibody, anti-TK1 antibody, anti-Gra | 0.974 | 0.947 | 0.983 |
| | antibody, anti-Her-2 antibody, TK1, CEA | | | |
| 28 | anti-ECPKA antibody, anti-TK1 antibody, anti-Gra | 0.971 | 0.947 | 0.978 |
| | antibody, anti-Her-2 antibody, TK1, CEA, AFP | | | |
| 29 | anti-ECPKA antibody, anti-TK1 antibody, anti-Gra | 0.974 | 0.961 | 0.978 |
| | antibody, anti-Her-2 antibody, TK1, CEA, AFP, CRP | | | |

As shown in Table 3, the biomarkers of Nos. 13, 24, and 25 had the highest accuracy, sensitivity, and specificity for diagnosis of bladder cancer. In particular, the biomarkers of No. 24 had the best accuracy, sensitivity, and specificity regardless of the presence or absence of NGAL. As shown in the biomarkers of No. 5, specificity and sensitivity was decreased even with the addition of P53. On the other hand, as shown in the biomarkers of No. 19, accuracy and specificity were enhanced when TK1 was added.

### 3. Breast Cancer-Specific Biomarker

TABLE 4

| No. | Combination of Biomarkers | Accuracy | Sensitivity | Specificity |
|---|---|---|---|---|
| 1 | GET (anti-ECPKA antibody, anti-TK1 antibody) | 0.796 | 0.960 | 0.761 |
| 2 | GET (anti-ECPKA antibody, anti-TK1 antibody), GHG | 0.821 | 0.940 | 0.796 |
| | (anti-Gra antibody, anti-Her-2 antibody) | | | |
| 3 | GET (anti-ECPKA antibody, anti-TK1 antibody), GHG | 0.825 | 0.980 | 0.791 |
| | (anti-Gra antibody, anti-Her-2 antibody), GCP | | | |
| | (anti-hP53 antibody, anti-c-myc antibody) | | | |
| 4 | GET (anti-ECPKA antibody, anti-TK1 antibody), GHG | 0.818 | 0.980 | 0.783 |
| | (anti-Gra antibody, anti-Her-2 antibody), GCP | | | |
| | (anti-hP53 antibody, anti-c-myc antibody), cP53 | | | |
| 5 | GET (anti-ECPKA antibody, anti-TK1 antibody), GHG | 0.821 | 0.940 | 0.796 |
| | (anti-Gra antibody, anti-Her-2 antibody), GCP | | | |
| | (anti-hP53 antibody, anti-c-myc antibody), cP53, hPKA | | | |
| 6 | GET (anti-ECPKA antibody, anti-TK1 antibody), GHG | 0.832 | 0.960 | 0.804 |
| | (anti-Gra antibody, anti-Her-2 antibody), GCP | | | |
| | (anti-hP53 antibody, anti-c-myc antibody), cP53, hPKA, | | | |
| | cTK1 | | | |
| 7 | GET (anti-ECPKA antibody, anti-TK1 antibody), GHG | 0.875 | 1.000 | 0.848 |
| | (anti-Gra antibody, anti-Her-2 antibody), GCP | | | |
| | (anti-hP53 antibody, anti-c-myc antibody), cP53, hPKA, | | | |
| | cTK1, AFP | | | |
| 8 | GET (anti-ECPKA antibody, anti-TK1 antibody), GHG | 0.875 | 1.000 | 0.848 |
| | (anti-Gra antibody, anti-Her-2 antibody), GCP | | | |
| | (anti-hP53 antibody, anti-c-myc antibody), cP53, hPKA, | | | |
| | cTK1, AFP, CEA | | | |
| 9 | GET (anti-ECPKA antibody, anti-TK1 antibody), GHG | 0.875 | 1.000 | 0.848 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA | | | |
| 10 | GET (anti-ECPKA antibody, anti-TK1 antibody), GHG | 0.875 | 1.000 | 0.848 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA, CRP | | | |
| 11 | GET (anti-ECPKA antibody, anti-TK1 antibody), GHG | 0.886 | 1.000 | 0.861 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA, CRP, B2M | | | |
| 12 | GET (anti-ECPKA antibody, anti-TK1 antibody), | 0.907 | 1.000 | 0.887 |
| | GHG (anti-Gra antibody, anti-Her-2 antibody), GCP | | | |
| | (anti-hP53 antibody, anti-c-myc antibody), cP53, | | | |
| | hPKA, cTK1, AFP, CEA, PSA, CRP, B2M, NGAL | | | |
| 13 | GET (anti-ECPKA antibody, anti-TK1 antibody), GHG | 0.900 | 1.000 | 0.878 |
| | (anti-Gra antibody, anti-Her-2 antibody), GCP | | | |
| | (anti-hP53 antibody, anti-c-myc antibody), cTK1, AFP, | | | |
| | CEA, CRP, B2M, NGAL | | | |
| 14 | GET (anti-ECPKA antibody, anti-TK1 antibody), GHG | 0.868 | 0.980 | 0.843 |
| | (anti-Gra antibody, anti-Her-2 antibody), GCP | | | |
| | (anti-hP53 antibody, anti-c-myc antibody), cTK1, AFP, | | | |
| | CRP | | | |
| 15 | GET (anti-ECPKA antibody, anti-TK1 antibody), AFP, | 0.825 | 0.940 | 0.800 |
| | CRP | | | |
| 16 | GET (anti-ECPKA antibody, anti-TK1 antibody), CRP | 0.793 | 0.940 | 0.761 |
| 17 | GHG (anti-Gra antibody, anti-Her-2 antibody), GCP | 0.900 | 1.000 | 0.878 |
| | (anti-hP53 antibody, anti-c-myc antibody), cP53, hPKA, | | | |
| | cTK1, AFP, CEA, PSA, CRP, B2M, NGAL | | | |
| 18 | GET (anti-ECPKA antibody, anti-TK1 antibody), GHG | 0.825 | 0.980 | 0.791 |
| | (anti-Gra antibody, anti-Her-2 antibody), GCP | | | |
| | (anti-hP53 antibody, anti-c-myc antibody), cTK1 | | | |
| 19 | GET (anti-ECPKA antibody, anti-TK1 antibody), GHG | 0.875 | 0.980 | 0.852 |
| | (anti-Gra antibody, anti-Her-2 antibody), GCP | | | |
| | (anti-hP53 antibody, anti-c-myc antibody), cTK1, AFP | | | |
| 20 | GET (anti-ECPKA antibody, anti-TK1 antibody), GHG | 0.875 | 0.980 | 0.852 |
| | (anti-Gra antibody, anti-Her-2 antibody), GCP | | | |
| | (anti-hP53 antibody, anti-c-myc antibody), cTK1, AFP, | | | |
| | CEA | | | |
| 21 | GET (anti-ECPKA antibody, anti-TK1 antibody), GHG | 0.875 | 0.980 | 0.852 |
| | (anti-Gra antibody, anti-Her-2 antibody), GCP | | | |
| | (anti-hP53 antibody, anti-c-myc antibody), cTK1, AFP, | | | |
| | CEA, PSA | | | |
| 22 | GET (anti-ECPKA antibody, anti-TK1 antibody), GHG | 0.875 | 0.980 | 0.852 |
| | (anti-Gra antibody, anti-Her-2 antibody), GCP | | | |
| | (anti-hP53 antibody, anti-c-myc antibody), cTK1, AFP, | | | |
| | CEA, PSA, CRP | | | |
| 23 | GET (anti-ECPKA antibody, anti-TK1 antibody), GHG | 0.882 | 1.000 | 0.857 |
| | (anti-Gra antibody, anti-Her-2 antibody), GCP | | | |
| | (anti-hP53 antibody, anti-c-myc antibody), cTK1, AFP, | | | |
| | CEA, PSA, CRP, B2M | | | |
| 24 | GET (anti-ECPKA antibody, anti-TK1 antibody), GHG | 0.900 | 1.000 | 0.878 |
| | (anti-Gra antibody, anti-Her-2 antibody), GCP | | | |
| | (anti-hP53 antibody, anti-c-myc antibody), cTK1, AFP, | | | |
| | CEA, PSA, CRP, B2M, NGAL | | | |
| 25 | GHG (anti-Gra antibody, anti-Her-2 antibody), GCP | 0.889 | 1.000 | 0.865 |
| | (anti-hP53 antibody, anti-c-myc antibody), cTK1, AFP, | | | |
| | CEA, PSA, CRP, B2M, NGAL | | | |
| 26 | anti-ECPKA antibody, anti-TK1 antibody, TK1 | 0.800 | 0.980 | 0.761 |
| 27 | anti-ECPKA antibody, anti-TK1 antibody, TK1, AFP | 0.821 | 0.940 | 0.796 |
| 28 | anti-ECPKA antibody, anti-TK1 antibody, anti-Gra | 0.875 | 1.000 | 0.848 |
| | antibody, anti-Her-2 antibody, TK1, AFP | | | |
| 29 | anti-ECPKA antibody, anti-TK1 antibody, anti-Gra | 0.868 | 1.000 | 0.839 |
| | antibody, anti-Her-2 antibody, TK1, AFP, B2M | | | |
| 30 | anti-ECPKA antibody, anti-TK1 antibody, anti-Gra | 0.893 | 1.000 | 0.870 |
| | antibody, anti-Her-2 antibody, TK1, AFP, B2M, NGAL | | | |
| 31 | GET (anti-ECPKA antibody, anti-TK1 antibody), GHG | 0.900 | 1.000 | 0.878 |
| | (anti-Gra antibody, anti-Her-2 antibody), GCP | | | |
| | (anti-hP53 antibody, anti-c-myc antibody), cP53, cTK1, | | | |
| | AFP, CEA, CRP, B2M, NGAL | | | |
| 32 | GET (anti-ECPKA antibody, anti-TK1 antibody), GHG | 0.900 | 1.000 | 0.878 |
| | (anti-Gra antibody, anti-Her-2 antibody), GCP | | | |
| | (anti-hP53 antibody, anti-c-myc antibody), hPKA, | | | |
| | cTK1, AFP, CEA, CRP, B2M, NGAL | | | |
| 33 | GET (anti-ECPKA antibody, anti-TK1 antibody), GHG | 0.904 | 1.000 | 0.883 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, CRP, B2M, NGAL | | | |

As shown in Table 4, the biomarkers of No. 12 had the highest accuracy, sensitivity, and specificity for diagnosis of breast cancer.

### 4. Cervical Cancer-Specific Biomarker

TABLE 5

| No. | Combination of Biomarkers | Accuracy | Sensitivity | Specificity |
|---|---|---|---|---|
| 1 | GET (anti-ECPKA antibody, anti-TK1 antibody) | 0.721 | 0.900 | 0.713 |
| 2 | GET (anti-ECPKA antibody, anti-TK1 antibody), GHG | 0.846 | 0.900 | 0.843 |
| | (anti-Gra antibody, anti-Her-2 antibody) | | | |
| 3 | GET (anti-ECPKA antibody, anti-TK1 antibody), GHG | 0.838 | 0.900 | 0.835 |
| | (anti-Gra antibody, anti-Her-2 antibody), GCP | | | |
| | (anti-hP53 antibody, anti-c-myc antibody) | | | |
| 4 | GET (anti-ECPKA antibody, anti-TK1 antibody), GHG | 0.867 | 0.900 | 0.865 |
| | (anti-Gra antibody, anti-Her-2 antibody), GCP | | | |
| | (anti-hP53 antibody, anti-c-myc antibody), cP53 | | | |
| 5 | GET (anti-ECPKA antibody, anti-TK1 antibody), GHG | 0.858 | 0.900 | 0.857 |
| | (anti-Gra antibody, anti-Her-2 antibody), GCP | | | |
| | (anti-hP53 antibody, anti-c-myc antibody), cP53, hPKA | | | |
| 6 | GET (anti-ECPKA antibody, anti-TK1 antibody), GHG | 0.863 | 0.900 | 0.861 |
| | (anti-Gra antibody, anti-Her-2 antibody), GCP | | | |
| | (anti-hP53 antibody, anti-c-myc antibody), cP53, hPKA, | | | |
| | cTK1 | | | |
| 7 | GET (anti-ECPKA antibody, anti-TK1 antibody), GHG | 0.892 | 1.000 | 0.887 |
| | (anti-Gra antibody, anti-Her-2 antibody), GCP | | | |
| | (anti-hP53 antibody, anti-c-myc antibody), cP53, hPKA, | | | |
| | cTK1, AFP | | | |
| 8 | GET (anti-ECPKA antibody, anti-TK1 antibody), GHG | 0.888 | 1.000 | 0.883 |
| | (anti-Gra antibody, anti-Her-2 antibody), GCP | | | |
| | (anti-hP53 antibody, anti-c-myc antibody), cP53, hPKA, | | | |
| | cTK1, AFP, CEA | | | |
| 9 | GET (anti-ECPKA antibody, anti-TK1 antibody), GHG | 0.888 | 1.000 | 0.883 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA | | | |
| 10 | GET (anti-ECPKA antibody, anti-TK1 antibody), GHG | 0.883 | 1.000 | 0.878 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA, CRP | | | |
| 11 | GET (anti-ECPKA antibody, anti-TK1 antibody), | 1.000 | 1.000 | 1.000 |
| | GHG (anti-Gra antibody, anti-Her-2 antibody), GCP | | | |
| | (anti-hP53 antibody, anti-c-myc antibody), cP53, | | | |
| | hPKA, cTK1, AFP, CEA, PSA, CRP, B2M | | | |
| 12 | GET (anti-ECPKA antibody, anti-TK1 antibody), GHG | 0.996 | 1.000 | 0.996 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA, CRP, B2M, NGAL | | | |
| 13 | GET (anti-ECPKA antibody, anti-TK1 antibody), | 1.000 | 1.000 | 1.000 |
| | GHG (anti-Gra antibody, anti-Her-2 antibody), GCP | | | |
| | (anti-hP53 antibody, anti-c-myc antibody), cTK1, | | | |
| | AFP, CEA, CRP, B2M, NGAL | | | |
| 14 | GET (anti-ECPKA antibody, anti-TK1 antibody), GHG | 0.892 | 1.000 | 0.887 |
| | (anti-Gra antibody, anti-Her-2 antibody), GCP | | | |
| | (anti-hP53 antibody, anti-c-myc antibody), cTK1, AFP, | | | |
| | CRP | | | |
| 15 | GET (anti-ECPKA antibody, anti-TK1 antibody), AFP, | 0.825 | 0.900 | 0.822 |
| | CRP | | | |
| 16 | GET (anti-ECPKA antibody, anti-TK1 antibody), CRP | 0.804 | 0.900 | 0.800 |
| 17 | GHG (anti-Gra antibody, anti-Her-2 antibody), GCP | 0.996 | 1.000 | 0.996 |
| | (anti-hP53 antibody, anti-c-myc antibody), cP53, hPKA, | | | |
| | cTK1, AFP, CEA, PSA, CRP, B2M, NGAL | | | |
| 18 | GET (anti-ECPKA antibody, anti-TK1 antibody), GHG | 0.838 | 0.900 | 0.835 |
| | (anti-Gra antibody, anti-Her-2 antibody), GCP | | | |
| | (anti-hP53 antibody, anti-c-myc antibody) | | | |
| 19 | GET (anti-ECPKA antibody, anti-TK1 antibody), GHG | 0.850 | 0.900 | 0.848 |
| | (anti-Gra antibody, anti-Her-2 antibody), GCP | | | |
| | (anti-hP53 antibody, anti-c-myc antibody), cTK1 | | | |
| 20 | GET (anti-ECPKA antibody, anti-TK1 antibody), GHG | 0.900 | 1.000 | 0.896 |
| | (anti-Gra antibody, anti-Her-2 antibody), GCP | | | |
| | (anti-hP53 antibody, anti-c-myc antibody), cTK1, AFP | | | |
| 21 | GET (anti-ECPKA antibody, anti-TK1 antibody), GHG | 0.896 | 1.000 | 0.891 |
| | (anti-Gra antibody, anti-Her-2 antibody), GCP | | | |
| | (anti-hP53 antibody, anti-c-myc antibody), cTK1, AFP, | | | |
| | CEA | | | |
| 22 | GET (anti-ECPKA antibody, anti-TK1 antibody), GHG | 0.892 | 1.000 | 0.887 |
| | (anti-Gra antibody, anti-Her-2 antibody), GCP | | | |
| | (anti-hP53 antibody, anti-c-myc antibody), cTK1, AFP, | | | |
| | CEA, PSA | | | |
| 23 | GET (anti-ECPKA antibody, anti-TK1 antibody), GHG | 0.888 | 1.000 | 0.883 |
| | (anti-Gra antibody, anti-Her-2 antibody), GCP | | | |
| | (anti-hP53 antibody, anti-c-myc antibody), cTK1, AFP, | | | |
| | CEA, PSA, CRP | | | |
| 24 | GET (anti-ECPKA antibody, anti-TK1 antibody), | 1.000 | 1.000 | 1.000 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M | | | |
| 25 | GET (anti-ECPKA antibody, anti-TK1 antibody), | 1.000 | 1.000 | 1.000 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M, NGAL | | | |
| 26 | GHG (anti-Gra antibody, anti-Her-2 antibody), GCP | 0.996 | 1.000 | 0.996 |
| | (anti-hP53 antibody, anti-c-myc antibody), cTK1, AFP, | | | |
| | CEA, PSA, CRP, B2M, NGAL | | | |
| 27 | anti-ECPKA antibody, anti-TK1 antibody | 0.721 | 0.900 | 0.713 |
| 28 | anti-ECPKA antibody, anti-TK1 antibody, AFP | 0.829 | 1.000 | 0.822 |
| 29 | anti-ECPKA antibody, anti-TK1 antibody, anti-Gra | 0.863 | 1.000 | 0.857 |
| | antibody, anti-Her-2 antibody, AFP | | | |
| 30 | anti-ECPKA antibody, anti-TK1 antibody, B2M | 0.988 | 1.000 | 0.987 |
| 31 | anti-ECPKA antibody, anti-TK1 antibody, AFP, B2M | 0.996 | 1.000 | 0.996 |
| 32 | anti-ECPKA antibody, anti-TK1 antibody, anti-Gra | 1.000 | 1.000 | 1.000 |
| | antibody, anti-Her-2 antibody, AFP, B2M | | | |

As shown in Table 5, the biomarkers of Nos. 11, 13, 24, 25, and 32 had the highest accuracy, sensitivity, and specificity for diagnosis of cervical cancer. In particular, it was confirmed that the biomarker of No. 32 had excellent accuracy, sensitivity, and specificity even with a combination of a small number of biomarkers.

### 5. Colorectal Cancer-Specific Biomarker

TABLE 6

| No. | Combination of Biomarkers | Accuracy | Sensitivity | Specificity |
|---|---|---|---|---|
| 1 | GET (anti-ECPKA antibody, anti-TK1 antibody) | 0.907 | 0.918 | 0.904 |
| 2 | GET (anti-ECPKA antibody, anti-TK1 antibody), GHG | 0.939 | 0.939 | 0.939 |
| | (anti-Gra antibody, anti-Her-2 antibody) | | | |
| 3 | GET (anti-ECPKA antibody, anti-TK1 antibody), GHG | 0.932 | 0.939 | 0.930 |
| | (anti-Gra antibody, anti-Her-2 antibody), GCP | | | |
| | (anti-hP53 antibody, anti-c-myc antibody) | | | |
| 4 | GET (anti-ECPKA antibody, anti-TK1 antibody), GHG | 0.939 | 0.959 | 0.935 |
| | (anti-Gra antibody, anti-Her-2 antibody), GCP | | | |
| | (anti-hP53 antibody, anti-c-myc antibody), cP53 | | | |
| 5 | GET (anti-ECPKA antibody, anti-TK1 antibody), GHG | 0.939 | 0.959 | 0.935 |
| | (anti-Gra antibody, anti-Her-2 antibody), GCP | | | |
| | (anti-hP53 antibody, anti-c-myc antibody), cP53, hPKA | | | |
| 6 | GET (anti-ECPKA antibody, anti-TK1 antibody), GHG | 0.943 | 0.959 | 0.939 |
| | (anti-Gra antibody, anti-Her-2 antibody), GCP | | | |
| | (anti-hP53 antibody, anti-c-myc antibody), cP53, hPKA, | | | |
| | cTK1 | | | |
| 7 | GET (anti-ECPKA antibody, anti-TK1 antibody), GHG | 0.928 | 0.959 | 0.922 |
| | (anti-Gra antibody, anti-Her-2 antibody), GCP | | | |
| | (anti-hP53 antibody, anti-c-myc antibody), cP53, hPKA, | | | |
| | cTK1, AFP | | | |
| 8 | GET (anti-ECPKA antibody, anti-TK1 antibody), GHG | 0.943 | 0.980 | 0.935 |
| | (anti-Gra antibody, anti-Her-2 antibody), GCP | | | |
| | (anti-hP53 antibody, anti-c-myc antibody), cP53, hPKA, | | | |
| | cTK1, AFP, CEA | | | |
| 9 | GET (anti-ECPKA antibody, anti-TK1 antibody), GHG | 0.935 | 0.959 | 0.930 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA | | | |
| 10 | GET (anti-ECPKA antibody, anti-TK1 antibody), GHG | 0.939 | 0.980 | 0.930 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA, CRP | | | |
| 11 | GET (anti-ECPKA antibody, anti-TK1 antibody), GHG | 0.943 | 0.959 | 0.939 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA, CRP, B2M | | | |
| 12 | GET (anti-ECPKA antibody, anti-TK1 antibody), GHG | 0.957 | 1.000 | 0.948 |
| | (anti-Gra antibody, anti-Her-2 antibody), GCP | | | |
| | (anti-hP53 antibody, anti-c-myc antibody), cP53, hPKA, | | | |
| | cTK1, AFP, CEA, PSA, CRP, B2M, NGAL | | | |
| 13 | GET (anti-ECPKA antibody, anti-TK1 antibody), GHG | 0.957 | 1.000 | 0.948 |
| | (anti-Gra antibody, anti-Her-2 antibody), GCP | | | |
| | (anti-hP53 antibody, anti-c-myc antibody), cTK1, AFP, | | | |
| | CEA, CRP, B2M, NGAL | | | |
| 14 | GET (anti-ECPKA antibody, anti-TK1 antibody), GHG | 0.939 | 0.980 | 0.930 |
| | (anti-Gra antibody, anti-Her-2 antibody), GCP | | | |
| | (anti-hP53 antibody, anti-c-myc antibody), cTK1, AFP, | | | |
| | CRP | | | |
| 15 | GET (anti-ECPKA antibody, anti-TK1 antibody), AFP, | 0.932 | 0.980 | 0.922 |
| | CRP | | | |
| 16 | GET (anti-ECPKA antibody, anti-TK1 antibody), CRP | 0.928 | 0.980 | 0.917 |
| 17 | GHG (anti-Gra antibody, anti-Her-2 antibody), GCP | 0.928 | 0.980 | 0.917 |
| | (anti-hP53 antibody, anti-c-myc antibody), cP53, hPKA, | | | |
| | cTK1, AFP, CEA, PSA, CRP, B2M, NGAL | | | |
| 18 | GET (anti-ECPKA antibody, anti-TK1 antibody), GHG | 0.932 | 0.939 | 0.930 |
| | (anti-Gra antibody, anti-Her-2 antibody), GCP | | | |
| | (anti-hP53 antibody, anti-c-myc antibody) | | | |
| 19 | GET (anti-ECPKA antibody, anti-TK1 antibody), GHG | 0.939 | 0.959 | 0.935 |
| | (anti-Gra antibody, anti-Her-2 antibody), GCP | | | |
| | (anti-hP53 antibody, anti-c-myc antibody), cTK1 | | | |
| 20 | GET (anti-ECPKA antibody, anti-TK1 antibody), GHG | 0.925 | 0.959 | 0.917 |
| | (anti-Gra antibody, anti-Her-2 antibody), GCP | | | |
| | (anti-hP53 antibody, anti-c-myc antibody), cTK1, AFP | | | |
| 21 | GET (anti-ECPKA antibody, anti-TK1 antibody), GHG | 0.939 | 0.959 | 0.935 |
| | (anti-Gra antibody, anti-Her-2 antibody), GCP | | | |
| | (anti-hP53 antibody, anti-c-myc antibody), cTK1, AFP, | | | |
| | CEA | | | |
| 22 | GET (anti-ECPKA antibody, anti-TK1 antibody), GHG | 0.932 | 0.939 | 0.930 |
| | (anti-Gra antibody, anti-Her-2 antibody), GCP | | | |
| | (anti-hP53 antibody, anti-c-myc antibody), cTK1, AFP, | | | |
| | CEA, PSA | | | |
| 23 | GET (anti-ECPKA antibody, anti-TK1 antibody), GHG | 0.943 | 0.980 | 0.935 |
| | (anti-Gra antibody, anti-Her-2 antibody), GCP | | | |
| | (anti-hP53 antibody, anti-c-myc antibody), cTK1, AFP, | | | |
| | CEA, PSA, CRP | | | |
| 24 | GET (anti-ECPKA antibody, anti-TK1 antibody), GHG | 0.943 | 0.980 | 0.935 |
| | (anti-Gra antibody, anti-Her-2 antibody), GCP | | | |
| | (anti-hP53 antibody, anti-c-myc antibody), cTK1, AFP, | | | |
| | CEA, PSA, CRP, B2M | | | |
| 25 | GET (anti-ECPKA antibody, anti-TK1 antibody), | 0.961 | 1.000 | 0.952 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M, NGAL | | | |
| 26 | GET (anti-ECPKA antibody, anti-TK1 antibody), GHG | 0.939 | 0.980 | 0.930 |
| | (anti-Gra antibody, anti-Her-2 antibody), GCP | | | |
| | (anti-hP53 antibody, anti-c-myc antibody), cTK1, AFP, | | | |
| | CRP | | | |
| 27 | GHG (anti-Gra antibody, anti-Her-2 antibody), GCP | 0.918 | 0.980 | 0.904 |
| | (anti-hP53 antibody, anti-c-myc antibody), cTK1, AFP, | | | |
| | CEA, PSA, CRP, B2M, NGAL | | | |
| 28 | anti-ECPKA antibody, anti-TK1 antibody, anti-Gra | 0.939 | 0.939 | 0.939 |
| | antibody, Her2 | | | |
| 29 | anti-ECPKA antibody, anti-TK1 antibody, anti-Gra | 0.935 | 0.939 | 0.935 |
| | antibody, anti-Her-2 antibody, AFP | | | |
| 30 | anti-ECPKA antibody, anti-TK1 antibody, anti-Gra | 0.946 | 0.959 | 0.943 |
| | antibody, anti-Her-2 antibody, AFP, CEA | | | |
| 31 | anti-ECPKA antibody, anti-TK1 antibody, anti-Gra | 0.939 | 0.980 | 0.930 |
| | antibody, anti-Her-2 antibody, AFP, CEA, CRP | | | |
| 32 | anti-ECPKA antibody, anti-TK1 antibody, anti-Gra | 0.964 | 1.000 | 0.957 |
| | antibody, anti-Her-2 antibody, AFP, CEA, CRP, | | | |
| | NGAL | | | |

As shown in Table 6, the biomarkers of No. 32 had the highest accuracy, sensitivity, and specificity for diagnosis of colorectal cancer. In addition, it was confirmed that the biomarkers of No. 25 had high accuracy, sensitivity, and specificity for diagnosis of colorectal cancer.

### 6. Gastric Cancer-Specific Biomarker

TABLE 7

| No. | Combination of Biomarkers | Accuracy | Sensitivity | Specificity |
|---|---|---|---|---|
| 1 | GET (anti-ECPKA antibody, anti-TK1 antibody) | 0.852 | 0.840 | 0.857 |
| 2 | GET (anti-ECPKA antibody, anti-TK1 antibody), | 0.897 | 0.870 | 0.909 |
| | GHG (anti-Gra antibody, anti-Her-2 antibody) | | | |
| 3 | GET (anti-ECPKA antibody, anti-TK1 antibody), | 0.906 | 0.900 | 0.909 |
| | GHG (anti-Gra antibody, anti-Her-2 antibody), GCP | | | |
| | (anti-hP53 antibody, anti-c-myc antibody) | | | |
| 4 | GET (anti-ECPKA antibody, anti-TK1 antibody), | 0.918 | 0.900 | 0.926 |
| | GHG (anti-Gra antibody, anti-Her-2 antibody), GCP | | | |
| | (anti-hP53 antibody, anti-c-myc antibody), cP53 | | | |
| 5 | GET (anti-ECPKA antibody, anti-TK1 antibody), | 0.939 | 0.930 | 0.943 |
| | GHG (anti-Gra antibody, anti-Her-2 antibody), GCP | | | |
| | (anti-hP53 antibody, anti-c-myc antibody), cP53, | | | |
| | hPKA | | | |
| 6 | GET (anti-ECPKA antibody, anti-TK1 antibody), | 0.939 | 0.940 | 0.939 |
| | GHG (anti-Gra antibody, anti-Her-2 antibody), GCP | | | |
| | (anti-hP53 antibody, anti-c-myc antibody), cP53, | | | |
| | hPKA, cTK1 | | | |
| 7 | GET (anti-ECPKA antibody, anti-TK1 antibody), | 0.936 | 0.920 | 0.943 |
| | GHG (anti-Gra antibody, anti-Her-2 antibody), GCP | | | |
| | (anti-hP53 antibody, anti-c-myc antibody), cP53, | | | |
| | hPKA, cTK1, AFP | | | |
| 8 | GET (anti-ECPKA antibody, anti-TK1 antibody), | 0.936 | 0.930 | 0.939 |
| | GHG (anti-Gra antibody, anti-Her-2 antibody), GCP | | | |
| | (anti-hP53 antibody, anti-c-myc antibody), cP53, | | | |
| | hPKA, cTK1, AFP, CEA | | | |
| 9 | GET (anti-ECPKA antibody, anti-TK1 antibody), | 0.933 | 0.920 | 0.939 |
| | GHG (anti-Gra antibody, anti-Her-2 antibody), GCP | | | |
| | (anti-hP53 antibody, anti-c-myc antibody), cP53, | | | |
| | hPKA, cTK1, AFP, CEA, PSA | | | |
| 10 | GET (anti-ECPKA antibody, anti-TK1 antibody), | 0.930 | 0.920 | 0.935 |
| | GHG (anti-Gra antibody, anti-Her-2 antibody), GCP | | | |
| | (anti-hP53 antibody, anti-c-myc antibody), cP53, | | | |
| | hPKA, cTK1, AFP, CEA, PSA, CRP | | | |
| 11 | GET (anti-ECPKA antibody, anti-TK1 antibody), | 0.942 | 0.930 | 0.948 |
| | GHG (anti-Gra antibody, anti-Her-2 antibody), | | | |
| | GCP (anti-hP53 antibody, anti-c-myc antibody), | | | |
| | cP53, hPKA, cTK1, AFP, CEA, PSA, CRP, B2M | | | |
| 12 | GET (anti-ECPKA antibody, anti-TK1 antibody), | 0.942 | 0.930 | 0.948 |
| | GHG (anti-Gra antibody, anti-Her-2 antibody), | | | |
| | GCP (anti-hP53 antibody, anti-c-myc antibody), | | | |
| | cP53, hPKA, cTK1, AFP, CEA, PSA, CRP, B2M, | | | |
| | NGAL | | | |
| 13 | GET (anti-ECPKA antibody, anti-TK1 antibody), | 0.927 | 0.920 | 0.930 |
| | GHG (anti-Gra antibody, anti-Her-2 antibody), GCP | | | |
| | (anti-hP53 antibody, anti-c-myc antibody), cTK1, | | | |
| | AFP, CEA, CRP, B2M, NGAL | | | |
| 14 | GET (anti-ECPKA antibody, anti-TK1 antibody), | 0.909 | 0.900 | 0.913 |
| | GHG (anti-Gra antibody, anti-Her-2 antibody), GCP | | | |
| | (anti-hP53 antibody, anti-c-myc antibody), cTK1, | | | |
| | AFP, CRP | | | |
| 15 | GET (anti-ECPKA antibody, anti-TK1 antibody), | 0.852 | 0.830 | 0.861 |
| | AFP, CRP | | | |
| 16 | GET (anti-ECPKA antibody, anti-TK1 antibody), | 0.861 | 0.830 | 0.874 |
| | CRP | | | |
| 17 | GHG (anti-Gra antibody, anti-Her-2 antibody), GCP | 0.924 | 0.920 | 0.926 |
| | (anti-hP53 antibody, anti-c-myc antibody), cP53, | | | |
| | hPKA, cTK1, AFP, CEA, PSA, CRP, B2M, NGAL | | | |
| 18 | GET (anti-ECPKA antibody, anti-TK1 antibody), | 0.906 | 0.900 | 0.909 |
| | GHG (anti-Gra antibody, anti-Her-2 antibody), GCP | | | |
| | (anti-hP53 antibody, anti-c-myc antibody) | | | |
| 19 | GET (anti-ECPKA antibody, anti-TK1 antibody), | 0.915 | 0.920 | 0.913 |
| | GHG (anti-Gra antibody, anti-Her-2 antibody), GCP | | | |
| | (anti-hP53 antibody, anti-c-myc antibody), cTK1 | | | |
| 20 | GET (anti-ECPKA antibody, anti-TK1 antibody), | 0.921 | 0.900 | 0.930 |
| | GHG (anti-Gra antibody, anti-Her-2 antibody), GCP | | | |
| | (anti-hP53 antibody, anti-c-myc antibody), cTK1, | | | |
| | AFP | | | |
| 21 | GET (anti-ECPKA antibody, anti-TK1 antibody), | 0.918 | 0.900 | 0.926 |
| | GHG (anti-Gra antibody, anti-Her-2 antibody), GCP | | | |
| | (anti-hP53 antibody, anti-c-myc antibody), cTK1, | | | |
| | AFP, CEA | | | |
| 22 | GET (anti-ECPKA antibody, anti-TK1 antibody), | 0.918 | 0.910 | 0.922 |
| | GHG (anti-Gra antibody, anti-Her-2 antibody), GCP | | | |
| | (anti-hP53 antibody, anti-c-myc antibody), cTK1, | | | |
| | AFP, CEA, PSA | | | |
| 23 | GET (anti-ECPKA antibody, anti-TK1 antibody), | 0.915 | 0.900 | 0.922 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP | | | |
| 24 | GET (anti-ECPKA antibody, anti-TK1 antibody), | 0.927 | 0.920 | 0.930 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M | | | |
| 25 | GET (anti-ECPKA antibody, anti-TK1 antibody), | 0.927 | 0.920 | 0.930 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M, NGAL | | | |
| 26 | GET (anti-ECPKA antibody, anti-TK1 antibody), | 0.927 | 0.920 | 0.930 |
| | GHG (anti-Gra antibody, anti-Her-2 antibody), GCP | | | |
| | (anti-hP53 antibody, anti-c-myc antibody), cTK1, | | | |
| | AFP, CEA, CRP, B2M, NGAL | | | |
| 27 | GET (anti-ECPKA antibody, anti-TK1 antibody), | 0.909 | 0.900 | 0.913 |
| | GHG (anti-Gra antibody, anti-Her-2 antibody), GCP | | | |
| | (anti-hP53 antibody, anti-c-myc antibody), cTK1, | | | |
| | AFP, CRP | | | |
| 28 | GHG (anti-Gra antibody, anti-Her-2 antibody), GCP | 0.885 | 0.880 | 0.887 |
| | (anti-hP53 antibody, anti-c-myc antibody), cTK1, | | | |
| | AFP, CEA, PSA, CRP, B2M, NGAL | | | |
| 29 | GET (anti-ECPKA antibody, anti-TK1 antibody), | 0.906 | 0.900 | 0.909 |
| | GHG (anti-Gra antibody, anti-Her-2 antibody), GCP | | | |
| | (anti-hP53 antibody, anti-c-myc antibody) | | | |
| 30 | GET (anti-ECPKA antibody, anti-TK1 antibody), | 0.909 | 0.890 | 0.917 |
| | GHG (anti-Gra antibody, anti-Her-2 antibody), GCP | | | |
| | (anti-hP53 antibody, anti-c-myc antibody), AFP | | | |
| 31 | GET (anti-ECPKA antibody, anti-TK1 antibody), | 0.921 | 0.900 | 0.930 |
| | GHG (anti-Gra antibody, anti-Her-2 antibody), GCP | | | |
| | (anti-hP53 antibody, anti-c-myc antibody), AFP, | | | |
| | TK1 | | | |
| 32 | GET (anti-ECPKA antibody, anti-TK1 antibody), | 0.909 | 0.880 | 0.922 |
| | GHG (anti-Gra antibody, anti-Her-2 antibody), GCP | | | |
| | (anti-hP53 antibody, anti-c-myc antibody), AFP, | | | |
| | PSA | | | |
| 33 | GET (anti-ECPKA antibody, anti-TK1 antibody), | 0.909 | 0.870 | 0.926 |
| | GHG (anti-Gra antibody, anti-Her-2 antibody), GCP | | | |
| | (anti-hP53 antibody, anti-c-myc antibody), AFP, | | | |
| | PSA, CRP | | | |
| 34 | GET (anti-ECPKA antibody, anti-TK1 antibody), | 0.906 | 0.870 | 0.922 |
| | GHG (anti-Gra antibody, anti-Her-2 antibody), GCP | | | |
| | (anti-hP53 antibody, anti-c-myc antibody), AFP, | | | |
| | PSA, CRP, NGAL | | | |

As shown in Table 7, the biomarkers of Nos. 11 and 12 had the highest accuracy, sensitivity, and specificity for diagnosis of gastric cancer. In addition, it was confirmed that the biomarkers of No. 11 had high accuracy, sensitivity, and specificity for diagnosis of gastric cancer regardless of whether NGAL is included.

### 7. Liver Cancer-Specific Biomarker

TABLE 8

| No. | Combination of Biomarkers | Accuracy | Sensitivity | Specificity |
|---|---|---|---|---|
| 1 | GET (anti-ECPKA antibody, anti-TK1 antibody) | 0.893 | 0.840 | 0.904 |
| 2 | GET (anti-ECPKA antibody, anti-TK1 antibody), GHG | 0.875 | 0.880 | 0.874 |
| | (anti-Gra antibody, anti-Her-2 antibody) | | | |
| 3 | GET (anti-ECPKA antibody, anti-TK1 antibody), GHG | 0.889 | 0.920 | 0.883 |
| | (anti-Gra antibody, anti-Her-2 antibody), GCP (anti-hP53 | | | |
| | antibody, anti-c-myc antibody) | | | |
| 4 | GET (anti-ECPKA antibody, anti-TK1 antibody), GHG | 0.911 | 0.920 | 0.909 |
| | (anti-Gra antibody, anti-Her-2 antibody), GCP (anti-hP53 | | | |
| | antibody, anti-c-myc antibody), cP53 | | | |
| 5 | GET (anti-ECPKA antibody, anti-TK1 antibody), GHG | 0.904 | 0.920 | 0.900 |
| | (anti-Gra antibody, anti-Her-2 antibody), GCP (anti-hP53 | | | |
| | antibody, anti-c-myc antibody), cP53, hPKA | | | |
| 6 | GET (anti-ECPKA antibody, anti-TK1 antibody), GHG | 0.907 | 0.920 | 0.904 |
| | (anti-Gra antibody, anti-Her-2 antibody), GCP (anti-hP53 | | | |
| | antibody, anti-c-myc antibody), cP53, hPKA, cTK1 | | | |
| 7 | GET (anti-ECPKA antibody, anti-TK1 antibody), GHG | 0.939 | 0.920 | 0.943 |
| | (anti-Gra antibody, anti-Her-2 antibody), GCP (anti-hP53 | | | |
| | antibody, anti-c-myc antibody), cP53, hPKA, cTK1, AFP | | | |
| 8 | GET (anti-ECPKA antibody, anti-TK1 antibody), GHG | 0.943 | 0.920 | 0.948 |
| | (anti-Gra antibody, anti-Her-2 antibody), GCP (anti-hP53 | | | |
| | antibody, anti-c-myc antibody), cP53, hPKA, cTK1, AFP, | | | |
| | CEA | | | |
| 9 | GET (anti-ECPKA antibody, anti-TK1 antibody), GHG | 0.939 | 0.920 | 0.943 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA | | | |
| 10 | GET (anti-ECPKA antibody, anti-TK1 antibody), | 0.946 | 0.940 | 0.948 |
| | GHG (anti-Gra antibody, anti-Her-2 antibody), GCP | | | |
| | (anti-hP53 antibody, anti-c-myc antibody), cP53, | | | |
| | hPKA, cTK1, AFP, CEA, PSA, CRP | | | |
| 11 | GET (anti-ECPKA antibody, anti-TK1 antibody), GHG | 0.936 | 0.920 | 0.939 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M | | | |
| 12 | GET (anti-ECPKA antibody, anti-TK1 antibody), GHG | 0.939 | 0.920 | 0.943 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M, NGAL | | | |
| 13 | GET (anti-ECPKA antibody, anti-TK1 antibody), GHG | 0.939 | 0.920 | 0.943 |
| | (anti-Gra antibody, anti-Her-2 antibody), GCP (anti-hP53 | | | |
| | antibody, anti-c-myc antibody), cTK1, AFP, CEA, CRP, | | | |
| | B2M, NGAL | | | |
| 14 | GET (anti-ECPKA antibody, anti-TK1 antibody), GHG | 0.936 | 0.920 | 0.939 |
| | (anti-Gra antibody, anti-Her-2 antibody), GCP (anti-hP53 | | | |
| | antibody, anti-c-myc antibody), cTK1, AFP, CRP | | | |
| 15 | GET (anti-ECPKA antibody, anti-TK1 antibody), AFP, | 0.925 | 0.880 | 0.935 |
| | CRP | | | |
| 16 | GET (anti-ECPKA antibody, anti-TK1 antibody), CRP | 0.879 | 0.920 | 0.870 |
| 17 | GHG (anti-Gra antibody, anti-Her-2 antibody), GCP | 0.936 | 0.920 | 0.939 |
| | (anti-hP53 antibody, anti-c-myc antibody), cP53, hPKA, | | | |
| | cTK1, AFP, CEA, PSA, CRP, B2M, NGAL | | | |
| 18 | GET (anti-ECPKA antibody, anti-TK1 antibody), GHG | 0.896 | 0.880 | 0.900 |
| | (anti-Gra antibody, anti-Her-2 antibody), GCP (anti-hP53 | | | |
| | antibody, anti-c-myc antibody), cTK1 | | | |
| 19 | GET (anti-ECPKA antibody, anti-TK1 antibody), GHG | 0.932 | 0.920 | 0.935 |
| | (anti-Gra antibody, anti-Her-2 antibody), GCP (anti-hP53 | | | |
| | antibody, anti-c-myc antibody), cTK1, AFP | | | |
| 20 | GET (anti-ECPKA antibody, anti-TK1 antibody), GHG | 0.932 | 0.920 | 0.935 |
| | (anti-Gra antibody, anti-Her-2 antibody), GCP (anti-hP53 | | | |
| | antibody, anti-c-myc antibody), cTK1, AFP, CEA | | | |
| 21 | GET (anti-ECPKA antibody, anti-TK1 antibody), GHG | 0.932 | 0.940 | 0.930 |
| | (anti-Gra antibody, anti-Her-2 antibody), GCP (anti-hP53 | | | |
| | antibody, anti-c-myc antibody), cTK1, AFP, CEA, PSA | | | |
| 22 | GET (anti-ECPKA antibody, anti-TK1 antibody), GHG | 0.936 | 0.920 | 0.939 |
| | (anti-Gra antibody, anti-Her-2 antibody), GCP (anti-hP53 | | | |
| | antibody, anti-c-myc antibody), cTK1, AFP, CEA, PSA, | | | |
| | CRP | | | |
| 23 | GET (anti-ECPKA antibody, anti-TK1 antibody), GHG | 0.939 | 0.940 | 0.939 |
| | (anti-Gra antibody, anti-Her-2 antibody), GCP (anti-hP53 | | | |
| | antibody, anti-c-myc antibody), cTK1, AFP, CEA, PSA, | | | |
| | CRP, B2M | | | |
| 24 | GET (anti-ECPKA antibody, anti-TK1 antibody), GHG | 0.939 | 0.920 | 0.943 |
| | (anti-Gra antibody, anti-Her-2 antibody), GCP (anti-hP53 | | | |
| | antibody, anti-c-myc antibody), cTK1, AFP, CEA, PSA, | | | |
| | CRP, B2M, NGAL | | | |
| 25 | GET (anti-ECPKA antibody, anti-TK1 antibody), GHG | 0.939 | 0.920 | 0.943 |
| | (anti-Gra antibody, anti-Her-2 antibody), GCP (anti-hP53 | | | |
| | antibody, anti-c-myc antibody), cTK1, AFP, CEA, CRP, | | | |
| | B2M, NGAL | | | |
| 26 | GET (anti-ECPKA antibody, anti-TK1 antibody), GHG | 0.936 | 0.920 | 0.939 |
| | (anti-Gra antibody, anti-Her-2 antibody), GCP (anti-hP53 | | | |
| | antibody, anti-c-myc antibody), cTK1, AFP, CRP | | | |
| 27 | GHG (anti-Gra antibody, anti-Her-2 antibody), GCP | 0.932 | 0.920 | 0.935 |
| | (anti-hP53 antibody, anti-c-myc antibody), cTK1, AFP, | | | |
| | CEA, PSA, CRP, B2M, NGAL | | | |
| 28 | GET (anti-ECPKA antibody, anti-TK1 antibody), GHG | 0.932 | 0.940 | 0.930 |
| | (anti-Gra antibody, anti-Her-2 antibody), GCP (anti-hP53 | | | |
| | antibody, anti-c-myc antibody), TK1, AFP, CEA, CRP, | | | |
| | B2M | | | |

As shown in Table 8, the biomarkers of No. 10 had the highest accuracy, sensitivity, and specificity for diagnosis of liver cancer.

### 8. Lung Cancer-Specific Biomarker

TABLE 9

| No. | Combination of Biomarkers | Accuracy | Sensitivity | Specificity |
|---|---|---|---|---|
| 1 | GET (anti-ECPKA antibody, anti-TK1 antibody) | 0.855 | 0.890 | 0.839 |
| 2 | GET (anti-ECPKA antibody, anti-TK1 antibody), GHG | 0.870 | 0.900 | 0.857 |
| | (anti-Gra antibody, anti-Her-2 antibody) | | | |
| 3 | GET (anti-ECPKA antibody, anti-TK1 antibody), | 0.876 | 0.900 | 0.865 |
| | GHG (anti-Gra antibody, anti-Her-2 antibody), GCP | | | |
| | (anti-hP53 antibody, anti-c-myc antibody) | | | |
| 4 | GET (anti-ECPKA antibody, anti-TK1 antibody), | 0.876 | 0.900 | 0.865 |
| | GHG (anti-Gra antibody, anti-Her-2 antibody), GCP | | | |
| | (anti-hP53 antibody, anti-c-myc antibody), cP53 | | | |
| 5 | GET (anti-ECPKA antibody, anti-TK1 antibody), GHG | 0.876 | 0.890 | 0.870 |
| | (anti-Gra antibody, anti-Her-2 antibody), GCP (anti-hP53 | | | |
| | antibody, anti-c-myc antibody), cP53, hPKA | | | |
| 6 | GET (anti-ECPKA antibody, anti-TK1 antibody), GHG | 0.876 | 0.890 | 0.870 |
| | (anti-Gra antibody, anti-Her-2 antibody), GCP (anti-hP53 | | | |
| | antibody, anti-c-myc antibody), cP53, hPKA, cTK1 | | | |
| 7 | GET (anti-ECPKA antibody, anti-TK1 antibody), GHG | 0.870 | 0.900 | 0.857 |
| | (anti-Gra antibody, anti-Her-2 antibody), GCP (anti-hP53 | | | |
| | antibody, anti-c-myc antibody), cP53, hPKA, cTK1, AFP | | | |
| 8 | GET (anti-ECPKA antibody, anti-TK1 antibody), GHG | 0.867 | 0.890 | 0.857 |
| | (anti-Gra antibody, anti-Her-2 antibody), GCP (anti-hP53 | | | |
| | antibody, anti-c-myc antibody), cP53, hPKA, cTK1, AFP, | | | |
| | CEA | | | |
| 9 | GET (anti-ECPKA antibody, anti-TK1 antibody), GHG | 0.870 | 0.890 | 0.861 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA | | | |
| 10 | GET (anti-ECPKA antibody, anti-TK1 antibody), GHG | 0.870 | 0.870 | 0.870 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP | | | |
| 11 | GET (anti-ECPKA antibody, anti-TK1 antibody), GHG | 0.873 | 0.870 | 0.874 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M | | | |
| 12 | GET (anti-ECPKA antibody, anti-TK1 antibody), GHG | 0.873 | 0.870 | 0.874 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M, NGAL | | | |
| 13 | GET (anti-ECPKA antibody, anti-TK1 antibody), GHG | 0.876 | 0.860 | 0.883 |
| | (anti-Gra antibody, anti-Her-2 antibody), GCP (anti-hP53 | | | |
| | antibody, anti-c-myc antibody), cTK1, AFP, CEA, CRP, | | | |
| | B2M, NGAL | | | |
| 14 | GET (anti-ECPKA antibody, anti-TK1 antibody), GHG | 0.867 | 0.860 | 0.870 |
| | (anti-Gra antibody, anti-Her-2 antibody), GCP (anti-hP53 | | | |
| | antibody, anti-c-myc antibody), cTK1, AFP, CRP | | | |
| 15 | GET (anti-ECPKA antibody, anti-TK1 antibody), AFP, | 0.858 | 0.900 | 0.839 |
| | CRP | | | |
| 16 | GET (anti-ECPKA antibody, anti-TK1 antibody), CRP | 0.855 | 0.880 | 0.843 |
| 17 | GHG (anti-Gra antibody, anti-Her-2 antibody), GCP | 0.864 | 0.890 | 0.852 |
| | (anti-hP53 antibody, anti-c-myc antibody), cP53, hPKA, | | | |
| | cTK1, AFP, CEA, PSA, CRP, B2M, NGAL | | | |
| 18 | GET (anti-ECPKA antibody, anti-TK1 antibody), | 0.876 | 0.910 | 0.861 |
| | GHG (anti-Gra antibody, anti-Her-2 antibody), GCP | | | |
| | (anti-hP53 antibody, anti-c-myc antibody), cTK1 | | | |
| 19 | GET (anti-ECPKA antibody, anti-TK1 antibody), GHG | 0.867 | 0.900 | 0.852 |
| | (anti-Gra antibody, anti-Her-2 antibody), GCP (anti-hP53 | | | |
| | antibody, anti-c-myc antibody), cTK1, AFP | | | |
| 20 | GET (anti-ECPKA antibody, anti-TK1 antibody), GHG | 0.870 | 0.900 | 0.857 |
| | (anti-Gra antibody, anti-Her-2 antibody), GCP (anti-hP53 | | | |
| | antibody, anti-c-myc antibody), cTK1, AFP, CEA | | | |
| 21 | GET (anti-ECPKA antibody, anti-TK1 antibody), GHG | 0.870 | 0.900 | 0.857 |
| | (anti-Gra antibody, anti-Her-2 antibody), GCP (anti-hP53 | | | |
| | antibody, anti-c-myc antibody), cTK1, AFP, CEA, PSA | | | |
| 22 | GET (anti-ECPKA antibody, anti-TK1 antibody), GHG | 0.873 | 0.870 | 0.874 |
| | (anti-Gra antibody, anti-Her-2 antibody), GCP (anti-hP53 | | | |
| | antibody, anti-c-myc antibody), cTK1, AFP, CEA, PSA, | | | |
| | CRP | | | |
| 23 | GET (anti-ECPKA antibody, anti-TK1 antibody), GHG | 0.876 | 0.860 | 0.883 |
| | (anti-Gra antibody, anti-Her-2 antibody), GCP (anti-hP53 | | | |
| | antibody, anti-c-myc antibody), cTK1, AFP, CEA, PSA, | | | |
| | CRP, B2M | | | |
| 24 | GET (anti-ECPKA antibody, anti-TK1 antibody), GHG | 0.876 | 0.860 | 0.883 |
| | (anti-Gra antibody, anti-Her-2 antibody), GCP (anti-hP53 | | | |
| | antibody, anti-c-myc antibody), cTK1, AFP, CEA, PSA, | | | |
| | CRP, B2M, NGAL | | | |
| 25 | GHG (anti-Gra antibody, anti-Her-2 antibody), GCP | 0.870 | 0.890 | 0.861 |
| | (anti-hP53 antibody, anti-c-myc antibody), cTK1, AFP, | | | |
| | CEA, PSA, CRP, B2M, NGAL | | | |
| 26 | GET (anti-ECPKA antibody, anti-TK1 antibody), | 0.876 | 0.910 | 0.861 |
| | GHG (anti-Gra antibody, anti-Her-2 antibody), GCP | | | |
| | (anti-hP53 antibody, anti-c-myc antibody), AFP | | | |
| 27 | GET (anti-ECPKA antibody, anti-TK1 antibody), GHG | 0.867 | 0.870 | 0.865 |
| | (anti-Gra antibody, anti-Her-2 antibody), GCP (anti-hP53 | | | |
| | antibody, anti-c-myc antibody), AFP, CRP | | | |
| 28 | GET (anti-ECPKA antibody, anti-TK1 antibody), GHG | 0.876 | 0.890 | 0.870 |
| | (anti-Gra antibody, anti-Her-2 antibody), GCP (anti-hP53 | | | |
| | antibody, anti-c-myc antibody), AFP, CRP, B2M | | | |

As shown in Table 9, the biomarkers of Nos. 3, 4, 18, and 26 had high accuracy, sensitivity, and specificity for diagnosis of lung cancer.

### 9. Ovarian Cancer-Specific Biomarker

TABLE 10

| No. | Combination of Biomarkers | Accuracy | Sensitivity | Specificity |
|---|---|---|---|---|
| 1 | GET (anti-ECPKA antibody, anti-TK1 antibody) | 0.774 | 0.909 | 0.761 |
| 2 | GET (anti-ECPKA antibody, anti-TK1 antibody), GHG | 0.794 | 0.909 | 0.783 |
| | (anti-Gra antibody, anti-Her-2 antibody) | | | |
| 3 | GET (anti-ECPKA antibody, anti-TK1 antibody), GHG | 0.810 | 0.955 | 0.796 |
| | (anti-Gra antibody, anti-Her-2 antibody), GCP (anti-hP53 | | | |
| | antibody, anti-c-myc antibody) | | | |
| 4 | GET (anti-ECPKA antibody, anti-TK1 antibody), GHG | 0.817 | 1.000 | 0.800 |
| | (anti-Gra antibody, anti-Her-2 antibody), GCP (anti-hP53 | | | |
| | antibody, anti-c-myc antibody), cP53 | | | |
| 5 | GET (anti-ECPKA antibody, anti-TK1 antibody), GHG | 0.813 | 0.955 | 0.800 |
| | (anti-Gra antibody, anti-Her-2 antibody), GCP (anti-hP53 | | | |
| | antibody, anti-c-myc antibody), cP53, hPKA | | | |
| 6 | GET (anti-ECPKA antibody, anti-TK1 antibody), GHG | 0.829 | 1.000 | 0.813 |
| | (anti-Gra antibody, anti-Her-2 antibody), GCP (anti-hP53 | | | |
| | antibody, anti-c-myc antibody), cP53, hPKA, cTK1 | | | |
| 7 | GET (anti-ECPKA antibody, anti-TK1 antibody), GHG | 0.833 | 1.000 | 0.817 |
| | (anti-Gra antibody, anti-Her-2 antibody), GCP (anti-hP53 | | | |
| | antibody, anti-c-myc antibody), cP53, hPKA, cTK1, AFP | | | |
| 8 | GET (anti-ECPKA antibody, anti-TK1 antibody), GHG | 0.833 | 1.000 | 0.817 |
| | (anti-Gra antibody, anti-Her-2 antibody), GCP (anti-hP53 | | | |
| | antibody, anti-c-myc antibody), cP53, hPKA, cTK1, AFP, | | | |
| | CEA | | | |
| 9 | GET (anti-ECPKA antibody, anti-TK1 antibody), GHG | 0.833 | 1.000 | 0.817 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA | | | |
| 10 | GET (anti-ECPKA antibody, anti-TK1 antibody), GHG | 0.865 | 1.000 | 0.852 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP | | | |
| 11 | GET (anti-ECPKA antibody, anti-TK1 antibody), | 0.905 | 1.000 | 0.896 |
| | GHG (anti-Gra antibody, anti-Her-2 antibody), GCP | | | |
| | (anti-hP53 antibody, anti-c-myc antibody), cP53, | | | |
| | hPKA, cTK1, AFP, CEA, PSA, CRP, B2M | | | |
| 12 | GET (anti-ECPKA antibody, anti-TK1 antibody), | 0.901 | 1.000 | 0.891 |
| | GHG (anti-Gra antibody, anti-Her-2 antibody), GCP | | | |
| | (anti-hP53 antibody, anti-c-myc antibody), cP53, | | | |
| | hPKA, cTK1, AFP, CEA, PSA, CRP, B2M, NGAL | | | |
| 13 | GET (anti-ECPKA antibody, anti-TK1 antibody), GHG | 0.889 | 1.000 | 0.878 |
| | (anti-Gra antibody, anti-Her-2 antibody), GCP (anti-hP53 | | | |
| | antibody, anti-c-myc antibody), cTK1, AFP, CEA, CRP, | | | |
| | B2M, NGAL | | | |
| 14 | GET (anti-ECPKA antibody, anti-TK1 antibody), GHG | 0.869 | 1.000 | 0.857 |
| | (anti-Gra antibody, anti-Her-2 antibody), GCP (anti-hP53 | | | |
| | antibody, anti-c-myc antibody), cTK1, AFP, CRP | | | |
| 15 | GET (anti-ECPKA antibody, anti-TK1 antibody), AFP, | 0.861 | 1.000 | 0.848 |
| | CRP | | | |
| 16 | GET (anti-ECPKA antibody, anti-TK1 antibody), CRP | 0.845 | 1.000 | 0.830 |
| 17 | GHG (anti-Gra antibody, anti-Her-2 antibody), GCP | 0.865 | 1.000 | 0.852 |
| | (anti-hP53 antibody, anti-c-myc antibody), cP53, hPKA, | | | |
| | cTK1, AFP, CEA, PSA, CRP, B2M, NGAL | | | |
| 18 | GET (anti-ECPKA antibody, anti-TK1 antibody), GHG | 0.810 | 0.955 | 0.796 |
| | (anti-Gra antibody, anti-Her-2 antibody), GCP (anti-hP53 | | | |
| | antibody, anti-c-myc antibody), cTK1 | | | |
| 19 | GET (anti-ECPKA antibody, anti-TK1 antibody), GHG | 0.810 | 0.955 | 0.796 |
| | (anti-Gra antibody, anti-Her-2 antibody), GCP (anti-hP53 | | | |
| | antibody, anti-c-myc antibody), cTK1, AFP | | | |
| 20 | GET (anti-ECPKA antibody, anti-TK1 antibody), GHG | 0.806 | 0.955 | 0.791 |
| | (anti-Gra antibody, anti-Her-2 antibody), GCP (anti-hP53 | | | |
| | antibody, anti-c-myc antibody), cTK1, AFP, CEA | | | |
| 21 | GET (anti-ECPKA antibody, anti-TK1 antibody), GHG | 0.806 | 0.955 | 0.791 |
| | (anti-Gra antibody, anti-Her-2 antibody), GCP (anti-hP53 | | | |
| | antibody, anti-c-myc antibody), cTK1, AFP, CEA, PSA | | | |
| 22 | GET (anti-ECPKA antibody, anti-TK1 antibody), GHG | 0.865 | 1.000 | 0.852 |
| | (anti-Gra antibody, anti-Her-2 antibody), GCP (anti-hP53 | | | |
| | antibody, anti-c-myc antibody), cTK1, AFP, CEA, PSA, | | | |
| | CRP | | | |
| 23 | GET (anti-ECPKA antibody, anti-TK1 antibody), GHG | 0.893 | 1.000 | 0.883 |
| | (anti-Gra antibody, anti-Her-2 antibody), GCP (anti-hP53 | | | |
| | antibody, anti-c-myc antibody), cTK1, AFP, CEA, PSA, | | | |
| | CRP, B2M | | | |
| 24 | GET (anti-ECPKA antibody, anti-TK1 antibody), GHG | 0.889 | 1.000 | 0.878 |
| | (anti-Gra antibody, anti-Her-2 antibody), GCP (anti-hP53 | | | |
| | antibody, anti-c-myc antibody), cTK1, AFP, CEA, PSA, | | | |
| | CRP, B2M, NGAL | | | |
| 25 | GHG (anti-Gra antibody, anti-Her-2 antibody), GCP | 0.813 | 0.955 | 0.800 |
| | (anti-hP53 antibody, anti-c-myc antibody), cTK1, AFP, | | | |
| | CEA, PSA, CRP, B2M, NGAL | | | |
| 26 | GET (anti-ECPKA antibody, anti-TK1 antibody), GHG | 0.802 | 0.955 | 0.787 |
| | (anti-Gra antibody, anti-Her-2 antibody), GCP (anti-hP53 | | | |
| | antibody, anti-c-myc antibody), AFP | | | |
| 27 | GET (anti-ECPKA antibody, anti-TK1 antibody), GHG | 0.790 | 0.955 | 0.774 |
| | (anti-Gra antibody, anti-Her-2 antibody), GCP (anti-hP53 | | | |
| | antibody, anti-c-myc antibody), AFP, CEA | | | |
| 28 | GET (anti-ECPKA antibody, anti-TK1 antibody), GHG | 0.869 | 1.000 | 0.857 |
| | (anti-Gra antibody, anti-Her-2 antibody), GCP (anti-hP53 | | | |
| | antibody, anti-c-myc antibody), AFP, CEA, CRP | | | |
| 29 | GET (anti-ECPKA antibody, anti-TK1 antibody), GHG | 0.893 | 1.000 | 0.883 |
| | (anti-Gra antibody, anti-Her-2 antibody), GCP (anti-hP53 | | | |
| | antibody, anti-c-myc antibody), AFP, CEA, CRP, B2M | | | |
| 30 | GET (anti-ECPKA antibody, anti-TK1 antibody), GHG | 0.885 | 1.000 | 0.874 |
| | (anti-Gra antibody, anti-Her-2 antibody), GCP (anti-hP53 | | | |
| | antibody, anti-c-myc antibody), AFP, TK1, PSA, CEA, | | | |
| | CRP, B2M | | | |

As shown in Table 10, the biomarkers of No. 11 had the highest accuracy, sensitivity, and specificity for diagnosis of ovarian cancer. In addition, the biomarkers of No. 12 had the decreased accuracy and specificity even with the addition of NGAL.

### 10. Pancreatic Cancer-Specific Biomarker

TABLE 11

| No. | Combination of Biomarkers | Accuracy | Sensitivity | Specificity |
|---|---|---|---|---|
| 1 | GET (anti-ECPKA antibody, anti-TK1 antibody) | 0.910 | 0.915 | 0.909 |
| 2 | GET (anti-ECPKA antibody, anti-TK1 antibody), GHG | 0.962 | 0.915 | 0.974 |
| | (anti-Gra antibody, anti-Her-2 antibody) | | | |
| 3 | GET (anti-ECPKA antibody, anti-TK1 antibody), GHG | 0.969 | 0.915 | 0.983 |
| | (anti-Gra antibody, anti-Her-2 antibody), GCP (anti-hP53 | | | |
| | antibody, anti-c-myc antibody) | | | |
| 4 | GET (anti-ECPKA antibody, anti-TK1 antibody), GHG | 0.962 | 0.915 | 0.974 |
| | (anti-Gra antibody, anti-Her-2 antibody), GCP (anti-hP53 | | | |
| | antibody, anti-c-myc antibody), cP53 | | | |
| 5 | GET (anti-ECPKA antibody, anti-TK1 antibody), GHG | 0.955 | 0.915 | 0.965 |
| | (anti-Gra antibody, anti-Her-2 antibody), GCP (anti-hP53 | | | |
| | antibody, anti-c-myc antibody), cP53, hPKA | | | |
| 6 | GET (anti-ECPKA antibody, anti-TK1 antibody), GHG | 0.972 | 0.915 | 0.987 |
| | (anti-Gra antibody, anti-Her-2 antibody), GCP (anti-hP53 | | | |
| | antibody, anti-c-myc antibody), cP53, hPKA, cTK1 | | | |
| 7 | GET (anti-ECPKA antibody, anti-TK1 antibody), GHG | 0.976 | 0.915 | 0.991 |
| | (anti-Gra antibody, anti-Her-2 antibody), GCP (anti-hP53 | | | |
| | antibody, anti-c-myc antibody), cP53, hPKA, cTK1, AFP | | | |
| 8 | GET (anti-ECPKA antibody, anti-TK1 antibody), GHG | 0.958 | 0.932 | 0.965 |
| | (anti-Gra antibody, anti-Her-2 antibody), GCP (anti-hP53 | | | |
| | antibody, anti-c-myc antibody), cP53, hPKA, cTK1, AFP, | | | |
| | CEA | | | |
| 9 | GET (anti-ECPKA antibody, anti-TK1 antibody), GHG | 0.958 | 0.932 | 0.965 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA | | | |
| 10 | GET (anti-ECPKA antibody, anti-TK1 antibody), GHG | 0.962 | 0.932 | 0.970 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP | | | |
| 11 | GET (anti-ECPKA antibody, anti-TK1 antibody), GHG | 0.976 | 0.949 | 0.983 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M | | | |
| 12 | GET (anti-ECPKA antibody, anti-TK1 antibody), GHG | 0.976 | 0.949 | 0.983 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M, NGAL | | | |
| 13 | GET (anti-ECPKA antibody, anti-TK1 antibody), | 0.986 | 0.966 | 0.991 |
| | GHG (anti-Gra antibody, anti-Her-2 antibody), GCP | | | |
| | (anti-hP53 antibody, anti-c-myc antibody), cTK1, | | | |
| | AFP, CEA, CRP, B2M, NGAL | | | |
| 14 | GET (anti-ECPKA antibody, anti-TK1 antibody), GHG | 0.972 | 0.915 | 0.987 |
| | (anti-Gra antibody, anti-Her-2 antibody), GCP (anti-hP53 | | | |
| | antibody, anti-c-myc antibody), cTK1, AFP, CRP | | | |
| 15 | GET (anti-ECPKA antibody, anti-TK1 antibody), AFP, | 0.924 | 0.915 | 0.926 |
| | CRP | | | |
| 16 | GET (anti-ECPKA antibody, anti-TK1 antibody), CRP | 0.920 | 0.915 | 0.922 |
| 17 | GHG (anti-Gra antibody, anti-Her-2 antibody), GCP | 0.931 | 0.949 | 0.926 |
| | (anti-hP53 antibody, anti-c-myc antibody), cP53, hPKA, | | | |
| | cTK1, AFP, CEA, PSA, CRP, B2M, NGAL | | | |
| 18 | GET (anti-ECPKA antibody, anti-TK1 antibody), GHG | 0.969 | 0.915 | 0.983 |
| | (anti-Gra antibody, anti-Her-2 antibody), GCP (anti-hP53 | | | |
| | antibody, anti-c-myc antibody) | | | |
| 19 | GET (anti-ECPKA antibody, anti-TK1 antibody), GHG | 0.976 | 0.915 | 0.991 |
| | (anti-Gra antibody, anti-Her-2 antibody), GCP (anti-hP53 | | | |
| | antibody, anti-c-myc antibody), cTK1 | | | |
| 20 | GET (anti-ECPKA antibody, anti-TK1 antibody), GHG | 0.976 | 0.915 | 0.991 |
| | (anti-Gra antibody, anti-Her-2 antibody), GCP (anti-hP53 | | | |
| | antibody, anti-c-myc antibody), cTK1, AFP | | | |
| 21 | GET (anti-ECPKA antibody, anti-TK1 antibody), GHG | 0.969 | 0.932 | 0.978 |
| | (anti-Gra antibody, anti-Her-2 antibody), GCP (anti-hP53 | | | |
| | antibody, anti-c-myc antibody), cTK1, AFP, CEA | | | |
| 22 | GET (anti-ECPKA antibody, anti-TK1 antibody), GHG | 0.969 | 0.932 | 0.978 |
| | (anti-Gra antibody, anti-Her-2 antibody), GCP (anti-hP53 | | | |
| | antibody, anti-c-myc antibody), cTK1, AFP, CEA, PSA | | | |
| 23 | GET (anti-ECPKA antibody, anti-TK1 antibody), GHG | 0.965 | 0.932 | 0.974 |
| | (anti-Gra antibody, anti-Her-2 antibody), GCP (anti-hP53 | | | |
| | antibody, anti-c-myc antibody), cTK1, AFP, CEA, PSA, | | | |
| | CRP | | | |
| 24 | GET (anti-ECPKA antibody, anti-TK1 antibody), GHG | 0.983 | 0.966 | 0.987 |
| | (anti-Gra antibody, anti-Her-2 antibody), GCP (anti-hP53 | | | |
| | antibody, anti-c-myc antibody), cTK1, AFP, CEA, PSA, | | | |
| | CRP, B2M | | | |
| 25 | GET (anti-ECPKA antibody, anti-TK1 antibody), | 0.986 | 0.966 | 0.991 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M, NGAL | | | |
| 26 | GHG (anti-Gra antibody, anti-Her-2 antibody), GCP | 0.934 | 0.949 | 0.930 |
| | (anti-hP53 antibody, anti-c-myc antibody), cTK1, AFP, | | | |
| | CEA, PSA, CRP, B2M, NGAL | | | |
| 27 | GET (anti-ECPKA antibody, anti-TK1 antibody), GHG | 0.965 | 0.915 | 0.978 |
| | (anti-Gra antibody, anti-Her-2 antibody), GCP (anti-hP53 | | | |
| | antibody, anti-c-myc antibody), AFP | | | |
| 28 | GET (anti-ECPKA antibody, anti-TK1 antibody), GHG | 0.958 | 0.915 | 0.970 |
| | (anti-Gra antibody, anti-Her-2 antibody), GCP (anti-hP53 | | | |
| | antibody, anti-c-myc antibody), AFP, CRP | | | |
| 29 | GET (anti-ECPKA antibody, anti-TK1 antibody), GHG | 0.969 | 0.915 | 0.983 |
| | (anti-Gra antibody, anti-Her-2 antibody), TK1, AFP, CRP | | | |
| 30 | GET (anti-ECPKA antibody, anti-TK1 antibody), GHG | 0.962 | 0.932 | 0.970 |
| | (anti-Gra antibody, anti-Her-2 antibody), AFP, CRP | | | |

As shown in Table 11, the biomarkers of Nos. 13 and 25 had the highest accuracy, sensitivity, and specificity for diagnosis of pancreatic cancer.

### 11. Prostate Cancer-Specific Biomarker

TABLE 12

| No. | Combination of Biomarkers | Accuracy | Sensitivity | Specificity |
|---|---|---|---|---|
| 1 | GET (anti-ECPKA antibody, anti-TK1 antibody) | 0.943 | 0.992 | 0.917 |
| 2 | GET (anti-ECPKA antibody, anti-TK1 antibody), GHG | 0.948 | 0.992 | 0.926 |
| | (anti-Gra antibody, anti-Her-2 antibody) | | | |
| 3 | GET (anti-ECPKA antibody, anti-TK1 antibody), GHG | 0.948 | 0.992 | 0.926 |
| | (anti-Gra antibody, anti-Her-2 antibody), GCP (anti-hP53 | | | |
| | antibody, anti-c-myc antibody) | | | |
| 4 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), cP53 | | | |
| 5 | GET (anti-ECPKA antibody, anti-TK1 antibody), GHG | 0.954 | 0.992 | 0.935 |
| | (anti-Gra antibody, anti-Her-2 antibody), GCP (anti-hP53 | | | |
| | antibody, anti-c-myc antibody), cP53, hPKA | | | |
| 6 | GET (anti-ECPKA antibody, anti-TK1 antibody), GHG | 0.954 | 0.992 | 0.935 |
| | (anti-Gra antibody, anti-Her-2 antibody), GCP (anti-hP53 | | | |
| | antibody, anti-c-myc antibody), cP53, hPKA, cTK1 | | | |
| 7 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), cP53, hPKA, cTK1, AFP | | | |
| 8 | GET (anti-ECPKA antibody, anti-TK1 antibody), GHG | 0.948 | 0.992 | 0.926 |
| | (anti-Gra antibody, anti-Her-2 antibody), GCP (anti-hP53 | | | |
| | antibody, anti-c-myc antibody), cP53, hPKA, cTK1, AFP, | | | |
| | CEA | | | |
| 9 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA | | | |
| 10 | GET (anti-ECPKA antibody, anti-TK1 antibody), GHG | 0.948 | 0.992 | 0.926 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP | | | |
| 11 | GET (anti-ECPKA antibody, anti-TK1 antibody), GHG | 0.954 | 0.992 | 0.935 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M | | | |
| 12 | GET (anti-ECPKA antibody, anti-TK1 antibody), GHG | 0.954 | 0.992 | 0.935 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M, NGAL | | | |
| 13 | GET (anti-ECPKA antibody, anti-TK1 antibody), | 0.957 | 0.992 | 0.939 |
| | GHG (anti-Gra antibody, anti-Her-2 antibody), GCP | | | |
| | (anti-hP53 antibody, anti-c-myc antibody), cTK1, | | | |
| | AFP, CEA, CRP, B2M, NGAL | | | |
| 14 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), cTK1, AFP, CRP | | | |
| 15 | GET (anti-ECPKA antibody, anti-TK1 antibody), AFP, | 0.943 | 0.992 | 0.917 |
| | CRP | | | |
| 16 | GET (anti-ECPKA antibody, anti-TK1 antibody), CRP | 0.940 | 0.992 | 0.913 |
| 17 | GHG (anti-Gra antibody, anti-Her-2 antibody), GCP | 0.957 | 0.992 | 0.939 |
| | (anti-hP53 antibody, anti-c-myc antibody), cP53, | | | |
| | hPKA, cTK1, AFP, CEA, PSA, CRP, B2M, NGAL | | | |
| 18 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), cTK1 | | | |
| 19 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), cTK1, AFP | | | |
| 20 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), cTK1, AFP, CEA | | | |
| 21 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), cTK1, AFP, CEA, PSA | | | |
| 22 | GET (anti-ECPKA antibody, anti-TK1 antibody), GHG | 0.948 | 0.992 | 0.926 |
| | (anti-Gra antibody, anti-Her-2 antibody), GCP (anti-hP53 | | | |
| | antibody, anti-c-myc antibody), cTK1, AFP, CEA, PSA, | | | |
| | CRP | | | |
| 23 | GET (anti-ECPKA antibody, anti-TK1 antibody), GHG | 0.954 | 0.992 | 0.935 |
| | (anti-Gra antibody, anti-Her-2 antibody), GCP (anti-hP53 | | | |
| | antibody, anti-c-myc antibody), cTK1, AFP, CEA, PSA, | | | |
| | CRP, B2M | | | |
| 24 | GET (anti-ECPKA antibody, anti-TK1 antibody), | 0.957 | 0.992 | 0.939 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M, NGAL | | | |
| 25 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), cTK1, AFP, CRP | | | |
| 26 | GHG (anti-Gra antibody, anti-Her-2 antibody), GCP | 0.951 | 0.992 | 0.930 |
| | (anti-hP53 antibody, anti-c-myc antibody), cTK1, AFP, | | | |
| | CEA, PSA, CRP, B2M, NGAL | | | |
| 27 | GET (anti-ECPKA antibody, anti-TK1 antibody), AFP | 0.946 | 0.992 | 0.922 |
| 28 | GET (anti-ECPKA antibody, anti-TK1 antibody), GHG | 0.948 | 0.992 | 0.926 |
| | (anti-Gra antibody, anti-Her-2 antibody), AFP | | | |
| 29 | GET (anti-ECPKA antibody, anti-TK1 antibody), AFP, | 0.943 | 0.992 | 0.917 |
| | PSA | | | |
| 30 | GET (anti-ECPKA antibody, anti-TK1 antibody), GHG | 0.948 | 0.992 | 0.926 |
| | (anti-Gra antibody, anti-Her-2 antibody), AFP, PSA | | | |
| 31 | GET (anti-ECPKA antibody, anti-TK1 antibody), GHG | 0.951 | 0.992 | 0.930 |
| | (anti-Gra antibody, anti-Her-2 antibody), GCP (anti-hP53 | | | |
| | antibody, anti-c-myc antibody), AFP, PSA | | | |

As shown in Table 12, the biomarkers of Nos. 13, 17, and 24 had the highest accuracy, sensitivity, and specificity for diagnosis of prostate cancer.

### 12. Renal Cancer-Specific Biomarker

TABLE 13

| No. | Combination of Biomarkers | Accuracy | Sensitivity | Specificity |
|---|---|---|---|---|
| 1 | GET (anti-ECPKA antibody, anti-TK1 antibody) | 0.868 | 0.913 | 0.852 |
| 2 | GET (anti-ECPKA antibody, anti-TK1 antibody), GHG | 0.942 | 0.950 | 0.939 |
| | (anti-Gra antibody, anti-Her-2 antibody) | | | |
| 3 | GET (anti-ECPKA antibody, anti-TK1 antibody), GHG | 0.939 | 0.963 | 0.930 |
| | (anti-Gra antibody, anti-Her-2 antibody), GCP (anti-hP53 | | | |
| | antibody, anti-c-myc antibody) | | | |
| 4 | GET (anti-ECPKA antibody, anti-TK1 antibody), GHG | 0.935 | 0.950 | 0.930 |
| | (anti-Gra antibody, anti-Her-2 antibody), GCP (anti-hP53 | | | |
| | antibody, anti-c-myc antibody), cP53 | | | |
| 5 | GET (anti-ECPKA antibody, anti-TK1 antibody), GHG | 0.935 | 0.950 | 0.930 |
| | (anti-Gra antibody, anti-Her-2 antibody), GCP (anti-hP53 | | | |
| | antibody, anti-c-myc antibody), cP53, hPKA | | | |
| 6 | GET (anti-ECPKA antibody, anti-TK1 antibody), GHG | 0.939 | 0.975 | 0.926 |
| | (anti-Gra antibody, anti-Her-2 antibody), GCP (anti-hP53 | | | |
| | antibody, anti-c-myc antibody), cP53, hPKA, cTK1 | | | |
| 7 | GET (anti-ECPKA antibody, anti-TK1 antibody), GHG | 0.932 | 0.950 | 0.926 |
| | (anti-Gra antibody, anti-Her-2 antibody), GCP (anti-hP53 | | | |
| | antibody, anti-c-myc antibody), cP53, hPKA, cTK1, AFP | | | |
| 8 | GET (anti-ECPKA antibody, anti-TK1 antibody), GHG | 0.939 | 0.938 | 0.939 |
| | (anti-Gra antibody, anti-Her-2 antibody), GCP (anti-hP53 | | | |
| | antibody, anti-c-myc antibody), cP53, hPKA, cTK1, AFP, | | | |
| | CEA | | | |
| 9 | GET (anti-ECPKA antibody, anti-TK1 antibody), GHG | 0.939 | 0.938 | 0.939 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA | | | |
| 10 | GET (anti-ECPKA antibody, anti-TK1 antibody), GHG | 0.935 | 0.950 | 0.930 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP | | | |
| 11 | GET (anti-ECPKA antibody, anti-TK1 antibody), GHG | 0.961 | 0.975 | 0.957 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M | | | |
| 12 | GET (anti-ECPKA antibody, anti-TK1 antibody), GHG | 0.961 | 0.988 | 0.952 |
| | (anti-Gra antibody, anti-Her-2 antibody), GCP (anti-hP53 | | | |
| | antibody, anti-c-myc antibody), cP53, hPKA, cTK1, AFP, | | | |
| | CEA, PSA, CRP, B2M, NGAL | | | |
| 13 | GET (anti-ECPKA antibody, anti-TK1 antibody), | 0.965 | 0.988 | 0.957 |
| | GHG (anti-Gra antibody, anti-Her-2 antibody), GCP | | | |
| | (anti-hP53 antibody, anti-c-myc antibody), cTK1, | | | |
| | AFP, CEA, CRP, B2M, NGAL | | | |
| 14 | GET (anti-ECPKA antibody, anti-TK1 antibody), GHG | 0.939 | 0.950 | 0.935 |
| | (anti-Gra antibody, anti-Her-2 antibody), GCP (anti-hP53 | | | |
| | antibody, anti-c-myc antibody), cTK1, AFP, CRP | | | |
| 15 | GET (anti-ECPKA antibody, anti-TK1 antibody), AFP, | 0.881 | 0.900 | 0.874 |
| | CRP | | | |
| 16 | GET (anti-ECPKA antibody, anti-TK1 antibody), CRP | 0.868 | 0.913 | 0.852 |
| 17 | GHG (anti-Gra antibody, anti-Her-2 antibody), GCP | 0.913 | 0.950 | 0.900 |
| | (anti-hP53 antibody, anti-c-myc antibody), cP53, hPKA, | | | |
| | cTK1, AFP, CEA, PSA, CRP, B2M, NGAL | | | |
| 18 | GET (anti-ECPKA antibody, anti-TK1 antibody), GHG | 0.942 | 0.988 | 0.926 |
| | (anti-Gra antibody, anti-Her-2 antibody), GCP (anti-hP53 | | | |
| | antibody, anti-c-myc antibody), cTK1 | | | |
| 19 | GET (anti-ECPKA antibody, anti-TK1 antibody), GHG | 0.939 | 0.975 | 0.926 |
| | (anti-Gra antibody, anti-Her-2 antibody), GCP (anti-hP53 | | | |
| | antibody, anti-c-myc antibody), cTK1, AFP | | | |
| 20 | GET (anti-ECPKA antibody, anti-TK1 antibody), GHG | 0.939 | 0.950 | 0.935 |
| | (anti-Gra antibody, anti-Her-2 antibody), GCP (anti-hP53 | | | |
| | antibody, anti-c-myc antibody), cTK1, AFP, CEA | | | |
| 21 | GET (anti-ECPKA antibody, anti-TK1 antibody), GHG | 0.935 | 0.950 | 0.930 |
| | (anti-Gra antibody, anti-Her-2 antibody), GCP (anti-hP53 | | | |
| | antibody, anti-c-myc antibody), cTK1, AFP, CEA, PSA | | | |
| 22 | GET (anti-ECPKA antibody, anti-TK1 antibody), GHG | 0.942 | 0.963 | 0.935 |
| | (anti-Gra antibody, anti-Her-2 antibody), GCP (anti-hP53 | | | |
| | antibody, anti-c-myc antibody), cTK1, AFP, CEA, PSA, | | | |
| | CRP | | | |
| 23 | GET (anti-ECPKA antibody, anti-TK1 antibody), GHG | 0.961 | 0.988 | 0.952 |
| | (anti-Gra antibody, anti-Her-2 antibody), GCP (anti-hP53 | | | |
| | antibody, anti-c-myc antibody), cTK1, AFP, CEA, PSA, | | | |
| | CRP, B2M | | | |
| 24 | GET (anti-ECPKA antibody, anti-TK1 antibody), | 0.965 | 0.988 | 0.957 |
| | GHG (anti-Gra antibody, anti-Her-2 antibody), GCP | | | |
| | (anti-hP53 antibody, anti-c-myc antibody), cTK1, | | | |
| | AFP, CEA, PSA, CRP, B2M, NGAL | | | |
| 25 | GHG (anti-Gra antibody, anti-Her-2 antibody), GCP | 0.906 | 0.975 | 0.883 |
| | (anti-hP53 antibody, anti-c-myc antibody), cTK1, AFP, | | | |
| | CEA, PSA, CRP, B2M, NGAL | | | |
| 26 | GET (anti-ECPKA antibody, anti-TK1 antibody), GHG | 0.942 | 0.963 | 0.935 |
| | (anti-Gra antibody, anti-Her-2 antibody), GCP (anti-hP53 | | | |
| | antibody, anti-c-myc antibody), AFP | | | |
| 27 | GET (anti-ECPKA antibody, anti-TK1 antibody), GHG | 0.935 | 0.950 | 0.930 |
| | (anti-Gra antibody, anti-Her-2 antibody), GCP (anti-hP53 | | | |
| | antibody, anti-c-myc antibody), AFP, CEA | | | |
| 28 | GET (anti-ECPKA antibody, anti-TK1 antibody), GHG | 0.926 | 0.938 | 0.922 |
| | (anti-Gra antibody, anti-Her-2 antibody), GCP (anti-hP53 | | | |
| | antibody, anti-c-myc antibody), AFP, CEA, CRP | | | |
| 29 | GET (anti-ECPKA antibody, anti-TK1 antibody), GHG | 0.952 | 0.975 | 0.943 |
| | (anti-Gra antibody, anti-Her-2 antibody), GCP (anti-hP53 | | | |
| | antibody, anti-c-myc antibody), AFP, CEA, CRP, B2M | | | |
| 30 | GET (anti-ECPKA antibody, anti-TK1 antibody), GHG | 0.952 | 0.975 | 0.943 |
| | (anti-Gra antibody, anti-Her-2 antibody), GCP (anti-hP53 | | | |
| | antibody, anti-c-myc antibody), AFP, CEA, CRP, B2M, | | | |
| | NGAL | | | |

As shown in Table 13, the biomarkers of Nos. 13 and 24 had the highest accuracy, sensitivity, and specificity for diagnosis of renal cancer. In particular, it was confirmed that the biomarkers of No. 13 had high accuracy, sensitivity, and specificity compared to the biomarkers of No. 24 even though PSA was excluded.

1.-20. (canceled)

21. A composition for diagnosing gastric cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and cTK1.

22. The composition for diagnosing gastric cancer according to claim 21, wherein the biomarker combination further comprises one or more biomarkers selected from the group consisting of cP53, hPKA, CEA, PSA, CRP, B2M, and NGAL.

23. The composition for diagnosing gastric cancer according to claim 21, wherein the biomarker combination comprises a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, and B2M; and
(ii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, CRP, B2M, and NGAL.

24. The composition for diagnosing gastric cancer according to claim 23, wherein the biomarker combination further comprises a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, and hPKA; and
(ii) anti-ECPKA antibody, anti-TK1 antibody, anti-granulin antibody, anti-Her-2 antibody, anti-hP53 antibody, anti-c-myc antibody, cP53, hPKA, cTK1, and AFP.

25. The composition for diagnosing gastric cancer according to claim 23, wherein the biomarker combination further comprises a biomarker combination selected from the group consisting of:
(i) anti-ECPKA antibody, anti-TK1 antibody, anti-granulin antibody, anti-Her-2 antibody, anti-hP53 antibody, anti-c-myc antibody, cP53, hPKA, and cTK1;
(ii) anti-ECPKA antibody, anti-TK1 antibody, anti-granulin antibody, anti-Her-2 antibody, anti-hP53 antibody, anti-c-myc antibody, cP53, hPKA, cTK1, AFP, and CEA;
(iii) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, and PSA;
(iv) anti-ECPKA antibody, anti-TK1 antibody, anti-granulin antibody, anti-Her-2 antibody, anti-hP53 antibody, anti-c-myc antibody, cP53, hPKA, cTK1, AFP, CEA, PSA, and CRP;
(v) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL;
(vi) anti-ECPKA antibody, anti-TK1 antibody, anti-granulin antibody, anti-Her-2 antibody, anti-hP53 antibody, anti-c-myc antibody, cTK1, and AFP;
(vii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, and B2M;
(viii) anti-ECPKA antibody, anti-TK1 antibody, anti-granulin antibody, anti-Her-2 antibody, anti-hP53 antibody, anti-c-myc antibody, cTK1, AFP, CEA, PSA, CRP, B2M, and NGAL;
(ix) anti-ECPKA antibody, anti-TK1 antibody, anti-granulin antibody, anti-Her-2 antibody, anti-hP53 antibody, anti-c-myc antibody, cTK1, AFP, CEA, CRP, B2M, and NGAL; and
(x) anti-ECPKA antibody, anti-TK1 antibody, anti-granulin antibody, anti-Her-2 antibody, anti-hP53 antibody, anti-c-myc antibody, AFP, and TK1.

26.-55. (canceled)

56. The composition according to claim 21, wherein the expression level is increased compared to the expression level of a normal control group.

57. The composition according to claim 21, wherein the agent is
an antibody or an antigen binding fragment thereof or an aptamer that specifically binds to the protein or fragment thereof; or
a nucleic acid comprising a polynucleotide identical to or complementary to a polynucleotide that encodes the protein or fragment thereof.

58. The composition according to claim 57, wherein the antibody is a polyclonal antibody or a monoclonal antibody.

59. The composition according to claim 57, wherein
the nucleic acid is a primer, a probe, an antisense oligonucleotide, or an aptamer.

60.-63. (canceled)

64. A kit for diagnosing gastric cancer, comprising:
an agent for measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and cTK1.

65.-74. (canceled)

75. A method for detecting a biomarker for diagnosing gastric cancer, comprising the steps of:
measuring the expression level of a gene or the expression level of a protein or a fragment thereof for a combination of anti-ECPKA antibody, anti-TK1 antibody, anti-granulin antibody, and anti-Her-2 antibody; and one or more biomarkers selected from the group consisting of anti-hP53 antibody, anti-c-myc antibody, AFP, and cTK1 in a biological sample isolated from a subject suspected of having gastric cancer; and
comparing the measured expression level with the expression level of a gene or the expression level of a protein or a fragment thereof in a normal control group.

76.-81. (canceled)

82. The method according to claim 75, wherein the biological sample is blood, plasma, serum, bone marrow fluid, lymph fluid, saliva, tear fluid, mucosal fluid, amniotic fluid, or a combination thereof.

83. The method according to claim 75, wherein the measuring step comprises the step of incubating the biological sample with an antibody or an antigen binding fragment thereof or an aptamer that specifically binds to the protein or fragment thereof, or incubating a polynucleotide identical to or complementary to a polynucleotide that encodes the gene.

84. The method according to claim 75, wherein the measuring step is performed by electrophoresis, immunoblotting, enzyme-linked immunosorbent assay (ELISA), polymerase chain reaction, Northern blotting, polymerase amplification reaction (polymerase chain reaction: PCR), protein chip, immunoprecipitation, microarray, electron microscopy, or a combination thereof.
